SEQUENCE LISTING (95 sequences)

SEQ ID NO: 1 - protein, 5 aa, synthetic construct
SGDLS

SEQ ID NO: 2 - protein, 6 aa, synthetic construct
YYNGEE

SEQ ID NO: 3 - protein, 11 aa, synthetic construct
ASSAAVHYGYT

SEQ ID NO: 4 - protein, 45 aa, synthetic construct
MGFRLLCCVAFCLLGAGPVDSGVTQTPKHLITATGQQVTLRCSPR

SEQ ID NO: 5 - protein, 17 aa, synthetic construct
VYWYQQSLDQGLQFLIQ

SEQ ID NO: 6 - protein, 37 aa, synthetic construct
RAKGNILERFSAQQFPDLHSELNLSSLELGDSALYFC

SEQ ID NO: 7 - protein, 10 aa, synthetic construct
FGSGTRLTVV

SEQ ID NO: 8 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 9 - protein, 304 aa, synthetic construct
MGFRLLCCVAFCLLGAGPVDSGVTQTPKHLITATGQQVTLRCSPRSGDLSVYWYQQSLDQGLQFLIQYYNGEERAKGNILERFSAQQFPDLHSELNLSSLELGDSALYFCASSAAVHYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 10 - protein, 7 aa, synthetic construct
TSDQSYG

SEQ ID NO: 11 - protein, 8 aa, synthetic construct
QGSYDEQN

SEQ ID NO: 12 - protein, 11 aa, synthetic construct
AMREQGAQKLV

SEQ ID NO: 13 - protein, 46 aa, synthetic construct
MSLSSLLKVVTASLWLGPGIAQKITQTQPGMFVQEKEAVTLDCTYD

SEQ ID NO: 14 - protein, 17 aa, synthetic construct
LFWYKQPSSGEMIFLIY

SEQ ID NO: 15 - protein, 34 aa, synthetic construct
ATEGRYSLNFQKARKSANLVISASQLGDSAMYFC

SEQ ID NO: 16 - protein, 11 aa, synthetic construct
FGQGTRLTINP

SEQ ID NO: 17 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 18 - protein, 271 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 19 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 20 - protein, 309 aa, synthetic construct
MGFRLLCCVAFCLLGAGPVDSGVTQTPKHLITATGQQVTLRCSPRSGDLSVYWYQQSLDQIGLQFLIQYYNGEERAKGNILERFSAQQFPDLHSELNLSSLELGDSALYFCASSAAVHYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - protein, 142 aa, synthetic construct
NIQNPDPAVYQLRDSKSSDKSVCLFTDFDSQTNVSQSKDSDVYITDKTVLDMRSMDFKSNSAIVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 276 aa, synthetic construct
MSLSSLLKVVTASLWLGPGIAQKITQTQPGMFVQEKEAVTLDCTYDTSDQSYGLFWYKQPSSIGEMIFLIYQGSYDEQNATEGRYSLNFQKARKSANLVISASQLGDSAMYFCAMREQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 5 aa, synthetic construct
MGHDK

SEQ ID NO: 24 - protein, 6 aa, synthetic construct
SYGVNS

SEQ ID NO: 25 - protein, 12 aa, synthetic construct
ASSPPGQPYEQY

SEQ ID NO: 26 - protein, 45 aa, synthetic construct
MTIRLLCYMGFYFLGAGLMEADIYQTPRYLVIGTGKKITLECSQT

SEQ ID NO: 27 - protein, 17 aa, synthetic construct
MYWYQQDPGMELHLIHY

SEQ ID NO: 28 - protein, 37 aa, synthetic construct
TEKGDLSSESTVSRIRTEHFPLTLESARPSHTSQYLC

SEQ ID NO: 29 - protein, 10 aa, synthetic construct
FGPGTRLTVT

SEQ ID NO: 30 - protein, 173 aa, synthetic construct; identical to SEQ ID NO: 8

SEQ ID NO: 31 - protein, 305 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSPPGQPYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 32 - protein, 6 aa, synthetic construct
VSGLRG

SEQ ID NO: 33 - protein, 7 aa, synthetic construct
LYSAGEE

SEQ ID NO: 34 - protein, 12 aa, synthetic construct
AVRMNTGFQKLV

SEQ ID NO: 35 - protein, 47 aa, synthetic construct
MEKMLECAFIVLWLQLGWLSGEDQVTQSPEALRLQEGESSSLNCSYT

SEQ ID NO: 36 - protein, 17 aa, synthetic construct
LFWYRQDPGKGPEFLFT

SEQ ID NO: 37 - protein, 32 aa, synthetic construct
KEKERLKATLTKKESFLHITAPKPEDSATYLC

SEQ ID NO: 38 - protein, 11 aa, synthetic construct
FGTGTRLLVSP

SEQ ID NO: 39 - protein, 137 aa, synthetic construct; identical to SEQ ID NO: 17

SEQ ID NO: 40 - protein, 269 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRMNTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 41 - protein, 177 aa, synthetic construct; identical to SEQ ID NO: 19

SEQ ID NO: 42 - protein, 309 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSPPGQPYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 43 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 273 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRMNTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 45 - protein, 134 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREQGAQKLVFGQGTRLTINP

SEQ ID NO: 46 - protein, 132 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRMNTGFQKLVFGTGTRLLVSP

SEQ ID NO: 47 - protein, 131 aa, synthetic construct
MGFRLLCCVAFCLLGAGPVDSGVTQTPKHLITATGQQVTLRCSPRSGDLSVYWYQQSLDQGLQFLIQYYNGEERAKGNILERFSAQQFPDLHSELNLSSLELGDSALYFCASSAAVHYGYTFGSGTRLTVV

SEQ ID NO: 48 - protein, 132 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSPPGQPYEQYFGPGTRLTVT

SEQ ID NO: 49 - DNA, 1812 bp, synthetic construct
  source 1..1812: mol_type = other DNA
atgggcttcaggctcctctgctgtgtggccttttgtctcctgggagcaggcccagtggattctggagtcacacaaaccccaaagcacctgatcacagcaactggacagcaagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgcggcagttcactatggctacaccttcggttcggggaccaggttaaccgttgtagaggacctgagaaacgtgacaccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctataaggagagcaattattcctactgtctgtctagccggctgagagtgagcgccacattttggcacaaccctcggaatcacttcagatgccaggtgcagtttcacggcctgtctgaggaggataagtggccagagggcagcccaaagccagtgacccagaacatctccgccgaggcatggggaagagcagactgtggcatcaccagcgcctcctaccagcagggcgtgctgtccgccacaatcctgtatgagatcctgctgggcaaggccaccctgtacgccgtgctggtgagcacactggtggtcatggctatggtgaagaggaagaactccagggcaaagcggagcggaagcggagccacaaatttctctctgctgaagcaggcaggcgatgtggaggagaaccctggaccaatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctcttctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagcagggagcccagaagctggtatttggccaaggaaccaggctgactatcaacccaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 50 - protein, 602 aa, synthetic construct
MGFRLLCCVAFCLLGAGPVDSGVTQTPKHLITATGQQVTLRCSPRSGDLSVYWYQQSLDQGLQFLIQYYNGEERAKGNILERFSAQQFPDLHSELNLSSLELGDSALYFCASSAAVHYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 51 - DNA, 1812 bp, synthetic construct
  source 1..1812: mol_type = other DNA
atgggcttccggctgctgtgctgcgtggcattttgcctgctgggagcaggacctgtggatagcggcgtgacccagacaccaaagcacctgatcaccgcaacaggacagcaggtgaccctgaggtgttccccaagatctggcgatctgtccgtgtactggtatcagcagtctctggaccagggcctgcagttcctgatccagtactataacggcgaggagcgggccaagggcaatatcctggagagattcagcgcccagcagtttcctgatctgcactccgagctgaatctgagctccctggagctgggcgactccgccctgtacttctgcgcctctagcgccgcagtgcactacggctatacctttggctctggcaccaggctgacagtggtggaggacctgcgcaatgtgacaccccctaaggtgtctctgttcgagcccagcaaggccgagatcgccaacaagcagaaggccaccctggtgtgcctggcccggggcttctttcctgatcacgtggagctgtcctggtgggtgaatggcaaggaggtgcactctggcgtgtgcaccgacccacaggcctacaaggagagcaactactcctattgtctgtcctctcggctgagagtgagcgccacattttggcacaaccctaggaatcacttccgctgccaggtgcagtttcacggcctgtctgaggaggataagtggccagagggaagcccaaagccagtgacccagaatatctccgccgaggcatggggaagggcagactgtggaatcacctctgccagctatcagcagggcgtgctgtccgccacaatcctgtacgagatcctgctgggcaaggccaccctgtatgccgtgctggtgtccacactggtggtcatggctatggtgaagaggaagaacagccgggccaagcggagcggatctggagccaccaacttcagcctgctgaagcaggcaggcgatgtggaggagaaccctggaccaatgtctctgagctccctgctgaaggtcgtgacagcaagcctgtggctgggaccaggaatcgcacagaagatcacccagacacagcctggcatgtttgtgcaggagaaggaggccgtgaccctggactgcacctacgacacatctgatcagagctacggcctgttctggtataagcagccatctagcggcgagatgatctttctgatctaccagggcagctatgatgagcagaacgccacagagggccggtactctctgaatttccagaaggccagaaagagcgccaacctggtcatcagcgcctcccagctgggcgactccgccatgtatttctgtgccatgagggagcagggcgcccagaagctggtgtttggccagggcacccgcctgacaatcaacccaaatatccagaatcccgagcctgccgtgtaccagctgaaggaccccagatcccaggattctaccctgtgcctgttcacagactttgatagccagatcaacgtgcccaagacaatggagtccggcaccttcatcacagacaagtgcgtgctggacatgaaggctatggactctaagagcaacggcgccatcgcctggagcaatcagacctccttcacatgccaggatatctttaaggagaccaacgccacatatccatcctctgacgtgccctgtgatgccaccctgacagagaagtccttcgagaccgacatgaacctgaattttcagaatctgctggtcatcgtgctgcggatcctgctgctgaaggtggccggctttaacctgctgatgacactgagactgtggagctcctgatga

SEQ ID NO: 52 - DNA, 1836 bp, Homo sapiens
  source 1..1836: mol_type = genomic DNA
atgggcttcaggctcctctgctgtgtggccttttgtctcctgggagcaggcccagtggattctggagtcacacaaaccccaaagcacctgatcacagcaactggacagcaagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgcggcagttcactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcagggcaaagcggagcggaagcggagccacaaatttctctctgctgaagcaggcaggcgatgtggaggagaaccctggaccaatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctcttctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagcagggagcccagaagctggtatttggccaaggaaccaggctgactatcaacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 53 - protein, 610 aa, Homo sapiens
MGFRLLCCVAFCLLGAGPVDSGVTQTPKHLITATGQQVTLRCSPRSGDLSVYWYQQSLDQGLQFLIQYYNGEERAKGNILERFSAQQFPDLHSELNLSSLELGDSALYFCASSAAVHYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRSGSGATNFSLLKQAGDVEENPGPMSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - DNA, 1836 bp, synthetic construct
  source 1..1836: mol_type = other DNA
atgggcttccggctgctgtgctgcgtggcattttgcctgctgggagcaggacctgtggatagcggcgtgacccagacaccaaagcacctgatcaccgcaacaggacagcaggtgaccctgaggtgttccccaagatctggcgatctgtccgtgtactggtatcagcagtctctggaccagggcctgcagttcctgatccagtactataacggcgaggagcgggccaagggcaatatcctggagagattcagcgcccagcagtttcctgatctgcactccgagctgaatctgagctccctggagctgggcgactccgccctgtacttctgcgcctctagcgccgcagtgcactacggctatacctttggctctggcaccaggctgacagtggtggaggacctgaataaggtgttcccccctgaggtggccgtgtttgagccttccgaggccgagatctctcacacccagaaggccaccctggtgtgcctggcaaccggcttctttccagatcacgtggagctgagctggtgggtgaacggcaaggaggtgcacagcggcgtgtccaccgacccacagcccctgaaggagcagcccgccctgaatgatagcagatattgcctgtctagccggctgagagtgtccgccaccttttggcagaaccctcggaatcacttcagatgtcaggtgcagttttacggcctgagcgagaatgacgagtggacccaggatagggccaagcccgtgacacagatcgtgtccgccgaggcatggggaagggcagactgcggcttcacatccgtgtcttatcagcagggcgtgctgagcgccaccatcctgtatgagatcctgctgggcaaggccacactgtacgccgtgctggtgtccgccctggtgctgatggctatggtgaagcggaaggactttcgggccaagcggagcggatctggagccaccaacttcagcctgctgaagcaggcaggcgatgtggaggagaaccctggaccaatgtctctgagctccctgctgaaggtcgtgacagcaagcctgtggctgggaccaggaatcgcacagaagatcacccagacacagcctggcatgtttgtgcaggagaaggaggccgtgaccctggactgcacctacgacacatctgatcagagctacggcctgttctggtataagcagccatctagcggcgagatgatctttctgatctaccagggcagctatgatgagcagaacgccacagagggccggtactctctgaatttccagaaggccagaaagagcgccaacctggtcatcagcgcctcccagctgggcgactccgccatgtatttctgtgccatgagggagcagggcgcccagaagctggtgtttggccagggcacccgcctgacaatcaacccaaacatccagaatcccgaccctgccgtgtatcagctgagggactccaagtcctctgataagagcgtgtgcctgttcaccgactttgattctcagacaaacgtgagccagtccaaggacagcgacgtgtacatcaccgacaagacagtgctggatatgcggagcatggacttcaagtctaacagcgccgtggcctggagcaataagtccgatttcgcctgcgccaatgcctttaacaattccatcatccctgaggataccttctttccatctcccgagagctcctgtgacgtgaagctggtggagaagtctttcgagaccgatacaaacctgaattttcagaacctgagcgtgatcggcttccggatcctgctgctgaaggtggccggcttcaatctgctgatgacactgagactgtggtctagctgatga

SEQ ID NO: 55 - DNA, 1809 bp, synthetic construct
  source 1..1809: mol_type = other DNA
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtccgccgggacagccttacgagcagtacttcgggccgggcaccaggctcacggtcacagaggacctgagaaacgtgacaccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctataaggagagcaattattcctactgtctgtctagccggctgagagtgagcgccacattttggcacaaccctcggaatcacttcagatgccaggtgcagtttcacggcctgtctgaggaggataagtggccagagggcagcccaaagccagtgacccagaacatctccgccgaggcatggggaagagcagactgtggcatcaccagcgcctcctaccagcagggcgtgctgtccgccacaatcctgtatgagatcctgctgggcaaggccaccctgtacgccgtgctggtgagcacactggtggtcatggctatggtgaagaggaagaactccagggcaaagcggagcggaagcggagccacaaatttctctctgctgaagcaggcaggcgatgtggaggagaaccctggaccaatggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcggatgaacacaggctttcagaaacttgtatttggaactggcacccgacttctggtcagtccaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 56 - protein, 601 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSPPGQPYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRMNTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 57 - DNA, 1809 bp, synthetic construct
  source 1..1809: mol_type = other DNA
atgaccatccggctgctgtgctacatgggcttctattttctgggcgccggcctgatggaggccgatatctaccagacccccagatacctggtcatcggcaccggcaagaagatcacactggagtgttcccagacaatgggccacgataagatgtactggtatcagcaggaccctggcatggagctgcacctgatccactactcctatggcgtgaactctaccgagaagggcgacctgagctccgagtccaccgtgtctcggatcagaacagagcacttcccactgaccctggagagcgccaggcctagccacacatcccagtacctgtgcgcctctagcccacctggacagccttacgagcagtatttcggcccaggcaccaggctgaccgtgacagaggatctgcgcaacgtgacaccacccaaggtgagcctgtttgagccctccaaggccgagatcgccaataagcagaaggccaccctggtgtgcctggcccgcggcttctttcctgatcacgtggagctgtcctggtgggtgaacggcaaggaggtgcactctggcgtgtgcaccgacccacaggcctataaggagtctaattacagctattgtctgtcctctaggctgcgcgtgtccgccacattctggcacaaccctaggaatcacttccgctgccaggtgcagtttcacggcctgtccgaggaggataagtggccagagggctctcctaagccagtgacccagaatatcagcgccgaggcatggggaagggcagactgtggaatcacctccgcctcttaccagcagggcgtgctgagcgccacaatcctgtacgagatcctgctgggcaaggccacactgtatgccgtgctggtgtccaccctggtggtcatggctatggtgaagcgcaagaactctcgggccaagagaagcggatccggagccacaaatttcagcctgctgaagcaggcaggcgatgtggaggagaacccaggacctatggagaagatgctggagtgcgcctttatcgtgctgtggctgcagctgggatggctgtctggagaggaccaggtgacccagagcccagaggccctgaggctgcaggagggagagagctcctctctgaattgtagctacacagtgtccggcctgcggggcctgttctggtatagacaggatcccggcaagggccctgagttcctgtttaccctgtactccgccggcgaggagaaggagaaggagagactgaaggccaccctgacaaagaaggagtcttttctgcacatcacagccccaaagcccgaggacagcgccacctatctgtgcgccgtgcggatgaacacaggcttccagaagctggtgtttggcaccggcacaagactgctggtgagcccaaacatccagaatcctgagccagccgtgtaccagctgaaggacccccggtcccaggattctaccctgtgcctgttcacagactttgatagccagatcaacgtgcccaagacaatggagtccggcaccttcatcacagacaagtgcgtgctggacatgaaggctatggactctaagagcaacggcgccatcgcctggtctaatcagaccagcttcacatgccaggatatctttaaggagaccaatgccacatacccaagctccgacgtgccctgtgatgccaccctgacagagaagagcttcgagaccgacatgaacctgaattttcagaacctgctggtcatcgtgctgaggatcctgctgctgaaggtggccggctttaatctgctgatgacactgcgcctgtggtctagctgatga

SEQ ID NO: 58 - DNA, 1833 bp, Homo sapiens
  source 1..1833: mol_type = genomic DNA
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtccgccgggacagccttacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcagggcaaagcggagcggaagcggagccacaaatttctctctgctgaagcaggcaggcgatgtggaggagaaccctggaccaatggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcggatgaacacaggctttcagaaacttgtatttggaactggcacccgacttctggtcagtccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 59 - protein, 609 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSPPGQPYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRSGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRMNTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - DNA, 1833 bp, synthetic construct
  source 1..1833: mol_type = other DNA
atgaccatccggctgctgtgctacatgggcttctattttctgggcgccggcctgatggaggccgatatctaccagacccccagatacctggtcatcggcaccggcaagaagatcacactggagtgttcccagacaatgggccacgataagatgtactggtatcagcaggaccctggcatggagctgcacctgatccactactcctatggcgtgaactctaccgagaagggcgacctgagctccgagtccaccgtgtctcggatcagaacagagcacttcccactgaccctggagagcgccaggcctagccacacatcccagtacctgtgcgcctctagcccacctggacagccttacgagcagtatttcggcccaggcaccaggctgaccgtgacagaggacctgaataaggtgttcccccctgaggtggccgtgtttgagccttccgaggccgagatctctcacacccagaaggccaccctggtgtgcctggcaaccggcttctttccagatcacgtggagctgagctggtgggtgaacggcaaggaggtgcacagcggcgtgtccaccgacccacagcccctgaaggagcagcccgccctgaatgatagcagatattgcctgtctagccggctgagagtgtccgccaccttttggcagaaccctcggaatcacttcagatgtcaggtgcagttttacggcctgagcgagaatgacgagtggacccaggatagggccaagcccgtgacacagatcgtgtccgccgaggcatggggaagggcagactgcggcttcacatccgtgtcttatcagcagggcgtgctgagcgccaccatcctgtatgagatcctgctgggcaaggccacactgtacgccgtgctggtgtccgccctggtgctgatggctatggtgaagcggaaggactttcgggccaagagaagcggatccggagccacaaatttcagcctgctgaagcaggcaggcgatgtggaggagaacccaggacctatggagaagatgctggagtgcgcctttatcgtgctgtggctgcagctgggatggctgtctggagaggaccaggtgacccagagcccagaggccctgaggctgcaggagggagagagctcctctctgaattgtagctacacagtgtccggcctgcggggcctgttctggtatagacaggatcccggcaagggccctgagttcctgtttaccctgtactccgccggcgaggagaaggagaaggagagactgaaggccaccctgacaaagaaggagtcttttctgcacatcacagccccaaagcccgaggacagcgccacctatctgtgcgccgtgcggatgaacacaggcttccagaagctggtgtttggcaccggcacaagactgctggtgagcccaaacatccagaatcccgaccctgccgtgtatcagctgagggactccaagtcctctgataagagcgtgtgcctgttcaccgactttgattctcagacaaacgtgagccagtccaaggacagcgacgtgtacatcaccgacaagacagtgctggatatgcggagcatggacttcaagtctaacagcgccgtggcctggagcaataagtccgatttcgcctgcgccaatgcctttaacaattccatcatccctgaggataccttctttccatctcccgagagctcctgtgacgtgaagctggtggagaagtctttcgagaccgatacaaacctgaattttcagaacctgagcgtgatcggcttccggatcctgctgctgaaggtggccggcttcaatctgctgatgacactgagactgtggtctagctgatga

SEQ ID NO: 61 - protein, 9 aa, synthetic construct
LAGVVNLPK

SEQ ID NO: 62 - protein, 9 aa, synthetic construct
LAGAVNLPK

SEQ ID NO: 63 - protein, 9 aa, synthetic construct
LAGCVNLPK

SEQ ID NO: 64 - protein, 9 aa, synthetic construct
LAGSVNLPK

SEQ ID NO: 65 - protein, 9 aa, Homo sapiens
LAGGVNLPK

SEQ ID NO: 66 - protein, 81 aa, synthetic construct
KESSKFPFGISPAQSHRNIKILEDEPPTVRMRPHCVPFWTGRILLPSAASVCPIPFEACHLCQAMTLRCPNTQGCCSSWAS

SEQ ID NO: 67 - protein, 81 aa, Homo sapiens
KESSKFPFGISPAQSHRNIKILEDEPHSKDETPLCTLLDWQDSLAKRCVCVSNTIRSLSFVPGNDFEMSKHPGLLLILGKL

SEQ ID NO: 68 - protein, 15 aa, synthetic construct
ILEDEPPTVRMRPHC

SEQ ID NO: 69 - protein, 22 aa, Porcine teschovirus-1
GSGATNFSLLKQAGDVEENPGP

SEQ ID NO: 70 - protein, 21 aa, Thosea asigna virus
GSGEGRGSLLTCGDVEENPGP

SEQ ID NO: 71 - protein, 23 aa, Equine rhinitis A virus
GSGQCTNYALLKLAGDVESNPGP

SEQ ID NO: 72 - protein, 25 aa, Foot-and-mouth disease virus
GSGVKQTLNFDLLKLAGDVESNPGP

SEQ ID NO: 73-76 - skipped (4 entries)

SEQ ID NO: 77 - protein, 4 aa, synthetic construct
RQKR

SEQ ID NO: 78 - DNA, 912 bp, synthetic construct
  source 1..912: mol_type = other DNA
atgggcttcaggctcctctgctgtgtggccttttgtctcctgggagcaggcccagtggattctggagtcacacaaaccccaaagcacctgatcacagcaactggacagcaagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgcggcagttcactatggctacaccttcggttcggggaccaggttaaccgttgtagaggacctgagaaacgtgacaccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctataaggagagcaattattcctactgtctgtctagccggctgagagtgagcgccacattttggcacaaccctcggaatcacttcagatgccaggtgcagtttcacggcctgtctgaggaggataagtggccagagggcagcccaaagccagtgacccagaacatctccgccgaggcatggggaagagcagactgtggcatcaccagcgcctcctaccagcagggcgtgctgtccgccacaatcctgtatgagatcctgctgggcaaggccaccctgtacgccgtgctggtgagcacactggtggtcatggctatggtgaagaggaagaactcc

SEQ ID NO: 79 - DNA, 819 bp, synthetic construct
  source 1..819: mol_type = other DNA
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctcttctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagcagggagcccagaagctggtatttggccaaggaaccaggctgactatcaacccaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 80 - DNA, 912 bp, synthetic construct
  source 1..912: mol_type = other DNA
atgggcttccggctgctgtgctgcgtggcattttgcctgctgggagcaggacctgtggatagcggcgtgacccagacaccaaagcacctgatcaccgcaacaggacagcaggtgaccctgaggtgttccccaagatctggcgatctgtccgtgtactggtatcagcagtctctggaccagggcctgcagttcctgatccagtactataacggcgaggagcgggccaagggcaatatcctggagagattcagcgcccagcagtttcctgatctgcactccgagctgaatctgagctccctggagctgggcgactccgccctgtacttctgcgcctctagcgccgcagtgcactacggctatacctttggctctggcaccaggctgacagtggtggaggacctgcgcaatgtgacaccccctaaggtgtctctgttcgagcccagcaaggccgagatcgccaacaagcagaaggccaccctggtgtgcctggcccggggcttctttcctgatcacgtggagctgtcctggtgggtgaatggcaaggaggtgcactctggcgtgtgcaccgacccacaggcctacaaggagagcaactactcctattgtctgtcctctcggctgagagtgagcgccacattttggcacaaccctaggaatcacttccgctgccaggtgcagtttcacggcctgtctgaggaggataagtggccagagggaagcccaaagccagtgacccagaatatctccgccgaggcatggggaagggcagactgtggaatcacctctgccagctatcagcagggcgtgctgtccgccacaatcctgtacgagatcctgctgggcaaggccaccctgtatgccgtgctggtgtccacactggtggtcatggctatggtgaagaggaagaacagc

SEQ ID NO: 81 - DNA, 819 bp, synthetic construct
  source 1..819: mol_type = other DNA
atgtctctgagctccctgctgaaggtcgtgacagcaagcctgtggctgggaccaggaatcgcacagaagatcacccagacacagcctggcatgtttgtgcaggagaaggaggccgtgaccctggactgcacctacgacacatctgatcagagctacggcctgttctggtataagcagccatctagcggcgagatgatctttctgatctaccagggcagctatgatgagcagaacgccacagagggccggtactctctgaatttccagaaggccagaaagagcgccaacctggtcatcagcgcctcccagctgggcgactccgccatgtatttctgtgccatgagggagcagggcgcccagaagctggtgtttggccagggcacccgcctgacaatcaacccaaatatccagaatcccgagcctgccgtgtaccagctgaaggaccccagatcccaggattctaccctgtgcctgttcacagactttgatagccagatcaacgtgcccaagacaatggagtccggcaccttcatcacagacaagtgcgtgctggacatgaaggctatggactctaagagcaacggcgccatcgcctggagcaatcagacctccttcacatgccaggatatctttaaggagaccaacgccacatatccatcctctgacgtgccctgtgatgccaccctgacagagaagtccttcgagaccgacatgaacctgaattttcagaatctgctggtcatcgtgctgcggatcctgctgctgaaggtggccggctttaacctgctgatgacactgagactgtggagctcctgatga

SEQ ID NO: 82 - DNA, 924 bp, Homo sapiens
  source 1..924: mol_type = genomic DNA
atgggcttcaggctcctctgctgtgtggccttttgtctcctgggagcaggcccagtggattctggagtcacacaaaccccaaagcacctgatcacagcaactggacagcaagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgcggcagttcactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 83 - DNA, 831 bp, Homo sapiens
  source 1..831: mol_type = genomic DNA
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctcttctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagcagggagcccagaagctggtatttggccaaggaaccaggctgactatcaacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 84 - DNA, 924 bp, synthetic construct
  source 1..924: mol_type = other DNA
atgggcttccggctgctgtgctgcgtggcattttgcctgctgggagcaggacctgtggatagcggcgtgacccagacaccaaagcacctgatcaccgcaacaggacagcaggtgaccctgaggtgttccccaagatctggcgatctgtccgtgtactggtatcagcagtctctggaccagggcctgcagttcctgatccagtactataacggcgaggagcgggccaagggcaatatcctggagagattcagcgcccagcagtttcctgatctgcactccgagctgaatctgagctccctggagctgggcgactccgccctgtacttctgcgcctctagcgccgcagtgcactacggctatacctttggctctggcaccaggctgacagtggtggaggacctgaataaggtgttcccccctgaggtggccgtgtttgagccttccgaggccgagatctctcacacccagaaggccaccctggtgtgcctggcaaccggcttctttccagatcacgtggagctgagctggtgggtgaacggcaaggaggtgcacagcggcgtgtccaccgacccacagcccctgaaggagcagcccgccctgaatgatagcagatattgcctgtctagccggctgagagtgtccgccaccttttggcagaaccctcggaatcacttcagatgtcaggtgcagttttacggcctgagcgagaatgacgagtggacccaggatagggccaagcccgtgacacagatcgtgtccgccgaggcatggggaagggcagactgcggcttcacatccgtgtcttatcagcagggcgtgctgagcgccaccatcctgtatgagatcctgctgggcaaggccacactgtacgccgtgctggtgtccgccctggtgctgatggctatggtgaagcggaaggacttt

SEQ ID NO: 85 - DNA, 831 bp, synthetic construct
  source 1..831: mol_type = other DNA
atgtctctgagctccctgctgaaggtcgtgacagcaagcctgtggctgggaccaggaatcgcacagaagatcacccagacacagcctggcatgtttgtgcaggagaaggaggccgtgaccctggactgcacctacgacacatctgatcagagctacggcctgttctggtataagcagccatctagcggcgagatgatctttctgatctaccagggcagctatgatgagcagaacgccacagagggccggtactctctgaatttccagaaggccagaaagagcgccaacctggtcatcagcgcctcccagctgggcgactccgccatgtatttctgtgccatgagggagcagggcgcccagaagctggtgtttggccagggcacccgcctgacaatcaacccaaacatccagaatcccgaccctgccgtgtatcagctgagggactccaagtcctctgataagagcgtgtgcctgttcaccgactttgattctcagacaaacgtgagccagtccaaggacagcgacgtgtacatcaccgacaagacagtgctggatatgcggagcatggacttcaagtctaacagcgccgtggcctggagcaataagtccgatttcgcctgcgccaatgcctttaacaattccatcatccctgaggataccttctttccatctcccgagagctcctgtgacgtgaagctggtggagaagtctttcgagaccgatacaaacctgaattttcagaacctgagcgtgatcggcttccggatcctgctgctgaaggtggccggcttcaatctgctgatgacactgagactgtggtctagctgatga

SEQ ID NO: 86 - DNA, 915 bp, Homo sapiens
  source 1..915: mol_type = genomic DNA
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtccgccgggacagccttacgagcagtacttcgggccgggcaccaggctcacggtcacagaggacctgagaaacgtgacaccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctataaggagagcaattattcctactgtctgtctagccggctgagagtgagcgccacattttggcacaaccctcggaatcacttcagatgccaggtgcagtttcacggcctgtctgaggaggataagtggccagagggcagcccaaagccagtgacccagaacatctccgccgaggcatggggaagagcagactgtggcatcaccagcgcctcctaccagcagggcgtgctgtccgccacaatcctgtatgagatcctgctgggcaaggccaccctgtacgccgtgctggtgagcacactggtggtcatggctatggtgaagaggaagaactcc

SEQ ID NO: 87 - DNA, 813 bp, Homo sapiens
  source 1..813: mol_type = genomic DNA
atggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcggatgaacacaggctttcagaaacttgtatttggaactggcacccgacttctggtcagtccaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 88 - DNA, 915 bp, synthetic construct
  source 1..915: mol_type = other DNA
atgaccatccggctgctgtgctacatgggcttctattttctgggcgccggcctgatggaggccgatatctaccagacccccagatacctggtcatcggcaccggcaagaagatcacactggagtgttcccagacaatgggccacgataagatgtactggtatcagcaggaccctggcatggagctgcacctgatccactactcctatggcgtgaactctaccgagaagggcgacctgagctccgagtccaccgtgtctcggatcagaacagagcacttcccactgaccctggagagcgccaggcctagccacacatcccagtacctgtgcgcctctagcccacctggacagccttacgagcagtatttcggcccaggcaccaggctgaccgtgacagaggatctgcgcaacgtgacaccacccaaggtgagcctgtttgagccctccaaggccgagatcgccaataagcagaaggccaccctggtgtgcctggcccgcggcttctttcctgatcacgtggagctgtcctggtgggtgaacggcaaggaggtgcactctggcgtgtgcaccgacccacaggcctataaggagtctaattacagctattgtctgtcctctaggctgcgcgtgtccgccacattctggcacaaccctaggaatcacttccgctgccaggtgcagtttcacggcctgtccgaggaggataagtggccagagggctctcctaagccagtgacccagaatatcagcgccgaggcatggggaagggcagactgtggaatcacctccgcctcttaccagcagggcgtgctgagcgccacaatcctgtacgagatcctgctgggcaaggccacactgtatgccgtgctggtgtccaccctggtggtcatggctatggtgaagcgcaagaactct

SEQ ID NO: 89 - DNA, 813 bp, synthetic construct
  source 1..813: mol_type = other DNA
atggagaagatgctggagtgcgcctttatcgtgctgtggctgcagctgggatggctgtctggagaggaccaggtgacccagagcccagaggccctgaggctgcaggagggagagagctcctctctgaattgtagctacacagtgtccggcctgcggggcctgttctggtatagacaggatcccggcaagggccctgagttcctgtttaccctgtactccgccggcgaggagaaggagaaggagagactgaaggccaccctgacaaagaaggagtcttttctgcacatcacagccccaaagcccgaggacagcgccacctatctgtgcgccgtgcggatgaacacaggcttccagaagctggtgtttggcaccggcacaagactgctggtgagcccaaacatccagaatcctgagccagccgtgtaccagctgaaggacccccggtcccaggattctaccctgtgcctgttcacagactttgatagccagatcaacgtgcccaagacaatggagtccggcaccttcatcacagacaagtgcgtgctggacatgaaggctatggactctaagagcaacggcgccatcgcctggtctaatcagaccagcttcacatgccaggatatctttaaggagaccaatgccacatacccaagctccgacgtgccctgtgatgccaccctgacagagaagagcttcgagaccgacatgaacctgaattttcagaacctgctggtcatcgtgctgaggatcctgctgctgaaggtggccggctttaatctgctgatgacactgcgcctgtggtctagctgatga

SEQ ID NO: 90 - DNA, 927 bp, Homo sapiens
  source 1..927: mol_type = genomic DNA
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtccgccgggacagccttacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 91 - DNA, 825 bp, Homo sapiens
  source 1..825: mol_type = genomic DNA
atggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcggatgaacacaggctttcagaaacttgtatttggaactggcacccgacttctggtcagtccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 92 - DNA, 927 bp, synthetic construct
  source 1..927: mol_type = other DNA
atgaccatccggctgctgtgctacatgggcttctattttctgggcgccggcctgatggaggccgatatctaccagacccccagatacctggtcatcggcaccggcaagaagatcacactggagtgttcccagacaatgggccacgataagatgtactggtatcagcaggaccctggcatggagctgcacctgatccactactcctatggcgtgaactctaccgagaagggcgacctgagctccgagtccaccgtgtctcggatcagaacagagcacttcccactgaccctggagagcgccaggcctagccacacatcccagtacctgtgcgcctctagcccacctggacagccttacgagcagtatttcggcccaggcaccaggctgaccgtgacagaggacctgaataaggtgttcccccctgaggtggccgtgtttgagccttccgaggccgagatctctcacacccagaaggccaccctggtgtgcctggcaaccggcttctttccagatcacgtggagctgagctggtgggtgaacggcaaggaggtgcacagcggcgtgtccaccgacccacagcccctgaaggagcagcccgccctgaatgatagcagatattgcctgtctagccggctgagagtgtccgccaccttttggcagaaccctcggaatcacttcagatgtcaggtgcagttttacggcctgagcgagaatgacgagtggacccaggatagggccaagcccgtgacacagatcgtgtccgccgaggcatggggaagggcagactgcggcttcacatccgtgtcttatcagcagggcgtgctgagcgccaccatcctgtatgagatcctgctgggcaaggccacactgtacgccgtgctggtgtccgccctggtgctgatggctatggtgaagcggaaggacttt

SEQ ID NO: 93 - DNA, 754 bp, synthetic construct
  source 1..754: mol_type = other DNA
ggtgacccagagcccagaggccctgaggctgcaggagggagagagctcctctctgaattgtagctacacagtgtccggcctgcggggcctgttctggtatagacaggatcccggcaagggccctgagttcctgtttaccctgtactccgccggcgaggagaaggagaaggagagactgaaggccaccctgacaaagaaggagtcttttctgcacatcacagccccaaagcccgaggacagcgccacctatctgtgcgccgtgcggatgaacacaggcttccagaagctggtgtttggcaccggcacaagactgctggtgagcccaaacatccagaatcccgaccctgccgtgtatcagctgagggactccaagtcctctgataagagcgtgtgcctgttcaccgactttgattctcagacaaacgtgagccagtccaaggacagcgacgtgtacatcaccgacaagacagtgctggatatgcggagcatggacttcaagtctaacagcgccgtggcctggagcaataagtccgatttcgcctgcgccaatgcctttaacaattccatcatccctgaggataccttctttccatctcccgagagctcctgtgacgtgaagctggtggagaagtctttcgagaccgatacaaacctgaattttcagaacctgagcgtgatcggcttccggatcctgctgctgaaggtggccggcttcaatctgctgatgacactgagactgtggtctagctgatga

SEQ ID NO: 94 - protein, 28 aa, synthetic construct
NIKILEDEPPTVRMRPHCVPFWTGRILL

SEQ ID NO: 95 - protein, 27 aa, Porcine teschovirus-1
RAKRSGSGATNFSLLKQAGDVEENPGP

(19) United States
(12) Patent Application Publication
(10) Pub. No.: US 2026/0265339 A1
(43) Pub. Date: Sep. 10, 2026

(54) T cell receptors Targeting Defective DNA Repair Proteins

(71) Applicant: University of Pittsburgh - Of the Commonwealth System of Higher Education, Pittsburgh, PA (US)

(72) Inventors: Udai Shankar Kammula, Pittsburgh, PA (US); Ghanshyam Singh Yadav, Pittsburgh, PA (US); Chetana Bhaskarla, Pittsburgh, PA (US)

(21) Appl. No.: 19/680,986

(22) Filed: May 18, 2026

Related U.S. Application Data
(62) Division of application No. 17/795,573, filed on Feb. 5, 2021.
(60) Provisional application No. 62/971,057, filed on Feb. 6, 2020.

(51) Int. Cl. C07K 14/705; A61P 35/00; C12N 5/0783; C12N 15/86

(52) U.S. Cl. CPC C07K 14/7051; A61P 35/00; C12N 5/0636; C12N 15/86

(57) ABSTRACT

This document provides isolated immune cells that include an exogenous T cell receptor (TCR) having affinity for an Ataxia-Telangiesctasia Mutated (ATM) peptide, an exogenous TCR having affinity for an AT-rich interactive domain-containing protein 1A (ARID1A) peptide, or both an exogenous TCR having affinity for an ATM peptide and an exogenous TCR having affinity for an ARID1A peptide, as well as methods and materials for making such immune cells. For example, isolated immune cells that included an exogenous TCR having affinity for a mutant ATM and/or exogenous TCR having affinity for an ARID1A peptide, methods and materials for making such immune cells, and methods and materials for using such immune cells to treat mammals (e.g., a human having cancer) are provided.

### CLAIM OF PRIORITY

This application is a divisional of U.S. application Ser. No. 17/795,573, filed Jul. 27, 2022, which is a national stage application under 35 U.S.C. § 371 of International Application No. PCT/US2021/016883, having an international filing date of Feb. 5, 2021, which claims priority to U.S. Provisional Application No. 62/971,057, filed on Feb. 6, 2020, the entire contents of which is hereby incorporated by reference.

### SEQUENCE LISTING

This application contains a Sequence Listing that has been submitted electronically as an XML file named “45049-0035002 SL ST26.XML.” The XML file, created on May 18, 2026, is 115,742 bytes in size. The material in the XML file is hereby incorporated by reference in its entirety.

### BACKGROUND

### 1. Technical Field

This document relates to isolated immune cells that include an exogenous T cell receptor (TCR) having affinity for an Ataxia-Telangiesctasia Mutated (ATM) peptide and/or an exogenous TCR having affinity for an AT-rich interactive domain-containing protein 1A (ARID1A) peptide, as well as methods and materials for making such immune cells. For example, this document provides isolated immune cells that included an exogenous TCR having affinity for a mutant ATM peptide and/or an exogenous TCR having affinity for a mutant ARID1A peptide, methods and materials for making such immune cells, and methods and materials for using such immune cells to treat mammals (e.g., a human having cancer).

### 2. Background Information

Defective DNA repair is a hallmark of cancer and results in genomic instability and accumulation of other genetic abnormalities. Ataxia-Telangiesctasia Mutated (ATM) is a PI3K-related serine/threonine protein kinase (PIKK) that plays a role in the repair of DNA double-strand breaks. The PIKK domain of ATM recognizes serine-glutamine and threonine-glutamine motifs of many proteins, including ones involved in cell-cycle checkpoint arrest (e.g., Chkl and Chk2), DNA repair (BRCA1 and RAD51), and apoptosis (p53). See, e.g., Choi, et al., Mol. Cancer Ther., 15 (8): 1781-91 (2016). The AT-rich interactive domain-containing protein 1A (ARID1A, also known as BAF250a) is a subunit of the SWI/SNF chromatin remodeling complex, which helps regulate gene transcription by changing chromatin structure. See, Bitler, et al., Nat. Cell Biol., 19 (8): 962-973 (2019). The gene encoding ATM and the gene encoding ARID1A are frequently mutated in a wide variety of cancers including ovarian, endometrial, kidney, pancreas, stomach, bladder, lung, breast, brain, and hematological malignancies.

### SUMMARY

This document is based, at least in part, on the discoveries of TCRs having affinity for an ATM (Ataxia-Telangiesctasia Mutated) peptide, e.g., a mutant ATM peptide, and TCRs having affinity for an ARID1A (AT-rich interactive domain-containing protein 1A) peptide, e.g., a mutant ARID1A peptide. As described herein, TCRs provided herein have an alpha chain and a beta chain. In some cases, a TCR provided herein can bind to a mutant ATM peptide (within the context of an MHC molecule such as HLA A*03:01). In some cases, a TCR provided herein can bind to a mutant ARID1A peptide (within the context of an MHC molecule such as HLA A*02:01).

In some cases, a TCR provided herein that has affinity for an ATM peptide can have an alpha chain that includes (i) the amino acid sequences set forth in SEQ ID NO: 10, SEQ ID NO:11, and SEQ ID NO:12, or (ii) the amino acid sequences set forth in SEQ ID NO:10 and SEQ ID NO:11 with no more than one amino acid modification, and the amino acid sequence set forth in SEQ ID NO: 12 with no more than two amino acid modifications, and a beta chain that includes (i) the amino acid sequences set forth in SEQ ID NO:1, SEQ ID NO:2, and SEQ ID NO:3, or (ii) the amino acid sequences set forth in SEQ ID NO: 1 and SEQ ID NO:2 with no more than one amino acid modification, and the amino acid sequence set forth in SEQ ID NO:3 with no more than two amino acid modifications. The amino acid modifications can be an amino acid substitution, an amino acid deletion, or an amino acid addition.

In some cases, a TCR provided herein that has affinity for an ARID1A peptide can have an alpha chain that includes (i) the amino acid sequences set forth in SEQ ID NO: 32, SEQ ID NO:33, and SEQ ID NO:34, or (ii) the amino acid sequences set forth in SEQ ID NO:32 and SEQ ID NO:33 with no more than one amino acid modification and the amino acid sequence set forth in SEQ ID NO:34 with no more than two amino acid modifications, and an beta chain that includes (i) the amino acid sequences set forth in SEQ ID NO:23, SEQ ID NO:24, and SEQ ID NO:25, or (ii) the amino acid sequences set forth in SEQ ID NO:23 and SEQ ID NO:24 with no more than one amino acid modification, and the amino acid sequence set forth in SEQ ID NO:25 with no more than two amino acid modifications. The amino acid modifications can be an amino acid substitution, an amino acid deletion, or an amino acid addition.

Immune cells (e.g., T cells) modified to include an exogenous TCR having affinity for an ATM peptide (e.g., a mutant ATM peptide), an exogenous TCR having affinity for an ARID1A peptide (e.g., a mutant ARID1A peptide), or both an exogenous TCR having affinity for an ATM peptide (e.g., a mutant ATM peptide) and an exogenous TCR having affinity for an ARID1A peptide (e.g., a mutant ARID1A peptide) as described herein can be used to treat a mammal having cancer. Mutations in ATM and ARID1A are commonly found, for example, in ovarian, endometrial, kidney, pancreas, gastric, bladder, lung, breast, brain, liver, cervical, uterine, prostate, or hematopoietic malignancies.

In one aspect, this document features an isolated immune cell (e.g., a T cell) comprising an exogenous TCR having affinity for an ATM peptide (e.g., a mutant ATM peptide comprising a valine, alanine, cysteine, or serine at the position corresponding to 2695 in the human ATM protein). The exogenous TCR includes an alpha chain and a beta chain, where the alpha chain includes (i) the amino acid sequences set forth in SEQ ID NO:10, SEQ ID NO:11, and SEQ ID NO:12, or (ii) the amino acid sequences set forth in SEQ ID NO: 10 and SEQ ID NO: 11 with no more than one amino acid modification and the amino acid sequence set forth in SEQ ID NO: 12 with no more than two amino acid modifications, wherein the amino acid modifications are selected from the group consisting of amino acid substitutions, amino acid deletions, and amino acid additions, and the beta chain includes (i) the amino acid sequences set forth in SEQ ID NO:1, SEQ ID NO:2, and SEQ ID NO:3, or (ii) the amino acid sequences set forth in SEQ ID NO:1 and SEQ ID NO:2 with no more than one amino acid modification and the amino acid sequence set forth in SEQ ID NO: 3 with no more than two amino acid modifications, wherein the amino acid modifications are selected from the group consisting of amino acid substitutions, amino acid deletions, and amino acid additions.

In some cases, the alpha chain includes an amino sequence having at least 80 percent identity, at least 90 percent identity, or at least 95 percent identity to the amino acid sequence set forth in SEQ ID NO:18 or SEQ ID NO:22. In some cases, the alpha chain comprises the amino acid sequence set forth in SEQ ID NO:10. In some cases, the alpha chain includes the amino acid sequence set forth in SEQ ID NO: 11. In some cases, the alpha chain includes the amino acid sequence set forth in SEQ ID NO:12. In some cases, the alpha chain includes the amino acid sequence set forth in SEQ ID NO: 10, SEQ ID NO: 11, and SEQ ID NO: 12.

In some cases, the beta chain includes an amino acid sequence having at least 80 percent identity, at least 90 percent identity, or at least 95 percent identity to the amino acid sequence set forth in SEQ ID NO:9 or SEQ ID NO:20. In some cases, the beta chain includes the amino acid sequence set forth in SEQ ID NO:1. In some cases, the beta chain includes the amino acid sequence set forth in SEQ ID NO:2. In some cases, the beta chain includes the amino acid sequence set forth in SEQ ID NO:3. In some cases, the beta chain comprises the amino acid sequence set forth in SEQ ID NO: 1, SEQ ID NO: 2 and SEQ ID NO:3.

In one aspect, this document features an isolated immune cell (e.g., a T cell) comprising an exogenous TCR having affinity for an ARID1A peptide (e.g., a mutant ARID1A peptide containing a frameshift at the position corresponding to 1960 in the human ARID1A protein). The exogenous TCR includes an alpha chain and a beta chain, where the alpha chain includes (i) the amino acid sequences set forth in SEQ ID NO: 32, SEQ ID NO:33, and SEQ ID NO:34, or (ii) the amino acid sequences set forth in SEQ ID NO:32 and SEQ ID NO:33 with no more than one amino acid modification and the amino acid sequence set forth in SEQ ID NO:34 with no more than two amino acid modifications, wherein the amino acid modifications are selected from the group consisting of amino acid substitutions, amino acid deletions, and amino acid additions, and the beta chain includes (i) the amino acid sequences set forth in SEQ ID NO:23, SEQ ID NO:24, and SEQ ID NO:25, or (ii) the amino acid sequences set forth in SEQ ID NO:23 and SEQ ID NO:24 with no more than one amino acid modification and the amino acid sequence set forth in SEQ ID NO:25 with no more than two amino acid modifications, wherein the amino acid modifications are selected from the group consisting of amino acid substitutions, amino acid deletions, and amino acid additions.

In some cases, the alpha chain includes an amino sequence having at least 80 percent identity, at least 90 percent identity, or at least 95 percent identity to the amino acid sequence set forth in SEQ ID NO:40 or SEQ ID NO:44. In some cases, the alpha chain includes the amino acid sequence set forth in SEQ ID NO:32. In some cases, the alpha chain includes the amino acid sequence set forth in SEQ ID NO:33. In some cases, the alpha chain includes the amino acid sequence set forth in SEQ ID NO:34. In some cases, the alpha chain includes the amino acid sequence set forth in SEQ ID NO: 32, SEQ ID NO:33, and SEQ ID NO:34.

In some cases, the beta chain includes an amino acid sequence having at least 80 percent identity, at least 90 percent identity, or at least 95 percent identity to the amino acid sequence set forth in SEQ ID NO:31 or SEQ ID NO:42. In some cases, the beta chain includes the amino acid sequence set forth in SEQ ID NO:23. In some cases, the beta chain includes the amino acid sequence set forth in SEQ ID NO:24. In some cases, the beta chain includes the amino acid sequence set forth in SEQ ID NO: 25. In some cases, the beta chain includes the amino acid sequence set forth in SEQ ID NO:23, SEQ ID NO: 24 and SEQ ID NO:25.

In any of the embodiments, the immune cell can be a T cell. Expression of the endogenous TCR alpha and beta chain coding sequences can be downregulated in the T cell.

This document also features a nucleic acid molecule that includes a nucleic acid sequence encoding an alpha chain of a TCR having affinity for an ATM peptide and a nucleic acid sequence encoding a beta chain of the TCR. In some cases, the alpha chain includes (i) the amino acid sequences set forth in SEQ ID NO:10, SEQ ID NO: 11, and SEQ ID NO:12, or (ii) the amino acid sequences set forth in SEQ ID NO: 10 and SEQ ID NO:11 with no more than one amino acid modification and the amino acid sequence set forth in SEQ ID NO: 12 with no more than two amino acid modifications, wherein the amino acid modifications are selected from the group consisting of amino acid substitutions, amino acid deletions, and amino acid additions, and the beta chain includes (i) the amino acid sequences set forth in SEQ ID NO: 1, SEQ ID NO:2, and SEQ ID NO:3, or (ii) the amino acid sequences set forth in SEQ ID NO:1 and SEQ ID NO:2 with no more than one amino acid modification and the amino acid sequence set forth in SEQ ID NO:3 with no more than two amino acid modifications, wherein the amino acid modifications are selected from the group consisting of amino acid substitutions, amino acid deletions, and amino acid additions.

In some cases, the alpha chain comprises an amino sequence having at least 80 percent identity, at least 90 percent identity, or at least 95 percent identity to the amino acid sequence set forth in SEQ ID NO:18 or SEQ ID NO:22. In some cases, the alpha chain includes the amino acid sequence set forth in SEQ ID NO:10. In some cases, the alpha chain includes the amino acid sequence set forth in SEQ ID NO:11. In some cases, the alpha chain includes the amino acid sequence set forth in SEQ ID NO: 12. In some cases, the alpha chain includes the amino acid sequence set forth in SEQ ID NO:10, SEQ ID NO: 11, and SEQ ID NO:12. In some cases, the beta chain comprises an amino acid sequence having at least 80 percent identity, at least 90 percent identity, or at least 95 percent identity to the amino acid sequence set forth in SEQ ID NO:9 or SEQ ID NO:20. In some cases, the beta chain includes the amino acid sequence set forth in SEQ ID NO:1. In some cases, the beta chain includes the amino acid sequence set forth in SEQ ID NO:2. In some cases, the beta chain includes the amino acid sequence set forth in SEQ ID NO:3. In some cases, the beta chain comprises the amino acid sequence set forth in SEQ ID NO:1, SEQ ID NO: 2 and SEQ ID NO:3.

This document also features a nucleic acid molecule that includes a nucleic acid sequence encoding an alpha chain of a TCR having affinity for an ARID1A peptide and a nucleic acid sequence encoding a beta chain of the TCR. In some cases, the alpha chain includes (i) the amino acid sequences set forth in SEQ ID NO:32, SEQ ID NO:33, and SEQ ID NO:34, or (ii) the amino acid sequences set forth in SEQ ID NO: 32 and SEQ ID NO:33 with no more than one amino acid modification and the amino acid sequence set forth in SEQ ID NO:34 with no more than two amino acid modifications, wherein the amino acid modifications are selected from the group consisting of amino acid substitutions, amino acid deletions, and amino acid additions; and the beta chain includes (i) the amino acid sequences set forth in SEQ ID NO: 23, SEQ ID NO:24, and SEQ ID NO:25, or (ii) the amino acid sequences set forth in SEQ ID NO:23 and SEQ ID NO:24 with no more than one amino acid modification and the amino acid sequence set forth in SEQ ID NO:25 with no more than two amino acid modifications, wherein the amino acid modifications are selected from the group consisting of amino acid substitutions, amino acid deletions, and amino acid additions.

In some cases, the alpha chain includes an amino sequence having at least 80 percent identity, at least 90 percent identity, or at least 95 percent identity to the amino acid sequence set forth in SEQ ID NO:40 or SEQ ID NO:44. In some cases, the alpha chain includes the amino acid sequence set forth in SEQ ID NO:32. In some cases, the alpha chain includes the amino acid sequence set forth in SEQ ID NO:33. In some cases, the alpha chain includes the amino acid sequence set forth in SEQ ID NO:34. In some cases, the alpha chain includes the amino acid sequence set forth in SEQ ID NO: 32, SEQ ID NO:33, and SEQ ID NO:34. In some cases, the beta chain includes an amino acid sequence having at least 80 percent identity, at least 90 percent identity, or at least 95 percent identity to the amino acid sequence set forth in SEQ ID NO:31 or SEQ ID NO:42. In some cases, the beta chain includes the amino acid sequence set forth in SEQ ID NO:23. In some cases, the beta chain includes the amino acid sequence set forth in SEQ ID NO:24. In some cases, the beta chain includes the amino acid sequence set forth in SEQ ID NO:25. In some cases, the beta chain includes the amino acid sequence set forth in SEQ ID NO:23, SEQ ID NO:24 and SEQ ID NO:25.

In any of the embodiments, the nucleic acid can include a promotor 5′ of the nucleic acid sequence encoding the alpha chain and/or can include a promoter 5′ of the nucleic acid sequence encoding the beta chain. The promoter can be a viral 5′ long terminal repeat or a viral 3′ long terminal repeat.

In any of the embodiments, the nucleic acid can be a vector, e.g., a viral vector such as a retroviral vector or a lentiviral vector.

In any of the embodiments, the nucleic acid further can include a nucleic acid sequence encoding a linker. In some cases, the linker can be 3′ of the nucleic acid sequence encoding the alpha chain and 5′ of the nucleic acid sequence encoding the beta chain. In some cases, the linker can be 3′ of the nucleic acid sequence encoding the beta chain and 5′ of the nucleic acid sequence encoding the alpha chain. In some cases, the linker is a self-cleaving peptide (e.g., a self-cleaving peptide of a foot-and-mouth disease virus (FMDV), an equine rhinitis A virus (ERAVO, a Thosea asigna virus (TaV), or a porcine tescho virus-1 (PTV-1)). In some cases, the self-cleaving peptide is a P2A peptide. In some cases, the linker includes a furin cleavage site.

This document also features a method of making an immune cell (e.g., a T cell) that includes an exogenous TCR having affinity for an ATM peptide and/or an exogenous TCR having affinity an ARID1A peptide. The method includes introducing, into the immune cell, a nucleic acid molecule that includes a nucleic acid sequence encoding an alpha chain of a TCR having affinity for an ATM peptide and a nucleic acid sequence encoding a beta chain of the TCR, and/or introducing a nucleic acid molecule that includes a nucleic acid sequence encoding an alpha chain of a TCR having affinity for an ARID1A peptide and a nucleic acid sequence encoding a beta chain of the TCR, wherein the exogenous TCR having affinity for the ATM peptide and/or the exogenous TCR having affinity for the ARID1A peptide is expressed from the nucleic acid in the immune cell.

In another aspect, this document features an isolated immune cell that includes a nucleic acid molecule and includes an exogenous TCR having affinity for an ATM peptide and/or an exogenous TCR having affinity for an ARID1A peptide. The nucleic acid molecule includes a nucleic acid sequence encoding an alpha chain of a TCR having affinity for an ATM peptide and a nucleic acid sequence encoding a beta chain of the TCR, and/or a nucleic acid sequence encoding an alpha chain of a TCR having affinity for an ARID1A peptide and a nucleic acid sequence encoding a beta chain of the TCR.

This document also features a population of cells that include at least one isolated immune cell, where the isolated immune cell includes a nucleic acid molecule and includes an exogenous TCR having affinity for an ATM peptide and/or an exogenous TCR having affinity for an ARID1A peptide. This document also features a pharmaceutical composition that includes the population of cells and a pharmaceutically acceptable carrier. A method for treating a mammal (e.g., a human) in need thereof also is provided that includes administering to the mammal an effective amount of the pharmaceutical composition. The mammal can have cancer (e.g., a cancer selected from the group consisting of melanoma, chronic lymphocytic leukemia, a myelodysplastic syndrome, and breast cancer). The immune cells can be autologous to the mammal.

In another aspect, this document features a method for providing a mammal (e.g., a human) with immune cells that include an exogenous TCR having affinity for an ATM peptide and/or an exogenous TCR having affinity for an ARID1A peptide. The method includes administering, to the mammal, the population of cells. The mammal can have cancer (e.g., ovarian, endometrial, kidney, pancreas, gastric, bladder, lung, breast, brain, liver, cervical, uterine, prostate, or a hematopoietic malignancy). The immune cells can be autologous to the mammal.

A method for providing a mammal (e.g., a human) with cells comprising a TCR having affinity for an ATM peptide and/or ARID1A peptide also is provided. The method includes delivering, to the mammal, a nucleic acid molecule that includes a nucleic acid sequence encoding an alpha chain of a TCR having affinity for an ATM peptide and a nucleic acid sequence encoding a beta chain of the TCR, and/or a nucleic acid sequence encoding an alpha chain of a TCR having affinity for an ARID1A peptide and a nucleic acid sequence encoding a beta chain of the TCR, wherein the nucleic acid is expressed in cells of the mammal. The mammal can have cancer (e.g., ovarian, endometrial, kidney, pancreas, gastric, bladder, lung, breast, brain, liver, cervical, uterine, prostate, or a hematopoietic malignancy). The immune cells can be autologous to the mammal.

In another aspect, this document features a method for treating a mammal (e.g., a human) having cancer. The method includes administering, to the mammal, a population of cells that include at least one isolated immune cell, where the isolated immune cell includes a nucleic acid molecule and includes an exogenous TCR having affinity for an ATM peptide and/or an exogenous TCR having affinity for an ARID1A peptide, or administering a nucleic acid molecule that includes a nucleic acid sequence encoding an alpha chain of a TCR having affinity for an ATM peptide and a nucleic acid sequence encoding a beta chain of the TCR, and/or a nucleic acid sequence encoding an alpha chain of a TCR having affinity for an ARID1A peptide and a nucleic acid sequence encoding a beta chain of the TCR. The immune cells can be autologous to the mammal.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A contains an alignment of mutant ATM peptides containing either a valine, alanine, cysteine, or serine at the position corresponding to 2695 (SEQ ID NOs: 61-64) in the human ATM protein and a wild-type ATM peptide containing a glycine at the position corresponding to 2695 (SEQ ID NO:65) in the human ATM protein (NCBI Reference Sequence: NP_000042.3).

FIG. 1B contains an alignment of a mutant ARID1A peptide containing a frameshift at the position corresponding to 1960 (1960fs) (SEQ ID NO:66) in the human ARID1A protein and a wild-type ARID1A peptide containing a histidine at the position corresponding to 1960 (SEQ ID NO:67) in the human ARID1A protein (NCBI Reference Sequence: NP_006006.3).

FIG. 1C contains the nucleotide sequence encoding the TRBV9*01+TRAV14/DV4 chimeric TCR (SEQ ID NO:49) and the amino acid sequence of the TRBV9*01+TRAV14/DV4 chimeric TCR (SEQ ID NO:50).

FIG. 2 contains an exemplary codon optimized nucleotide sequence encoding the TRBV9*01+TRAV14/DV4 chimeric TCR (SEQ ID NO:51) and the amino acid sequence of the TRBV9*01+TRAV14/DV4 chimeric TCR (SEQ ID NO:50).

FIG. 3 contains the nucleotide sequence encoding a human TRBV9*01+TRAV14/DV4 TCR (SEQ ID NO:52) and the amino acid sequence of the human TRBV9*01+TRAV14/DV4 TCR (SEQ ID NO:53).

FIG. 4 contains an exemplary codon optimized nucleotide sequence encoding the human TRBV9*01+TRAV14/DV4 TCR (SEQ ID NO:54) and the amino acid sequence of the human TRBV9*01+TRAV14/DV4 TCR (SEQ ID NO:53).

FIG. 5 contains the nucleotide sequence encoding a TRBV25-1+TRAV20 chimeric TCR (SEQ ID NO:55) and the amino acid sequence of the TRBV25-1+TRAV20chimeric TCR (SEQ ID NO:56).

FIG. 6 contains an exemplary codon optimized nucleotide sequence encoding the TRBV25-1+TRAV20 chimeric TCR (SEQ ID NO:57) and the amino acid sequence of the TRBV25-1+TRAV20 chimeric TCR (SEQ ID NO:56).

FIG. 7 contains the nucleotide sequence encoding the human TRBV25-1+TRAV20 (SEQ ID NO:58) TCR and the amino acid sequence of the human TRBV25-1+TRAV20 TCR (SEQ ID NO:59).

FIG. 8 contains an exemplary codon optimized nucleotide sequence encoding a human TRBV25-1+TRAV20 TCR (SEQ ID NO:60) and the amino acid sequence of the human TRBV25-1+TRAV20 TCR (SEQ ID NO:59).

FIG. 9A contains a graph of interferon gamma (IFNγ) (pg/mL) in the supernatant after TCR-transduced T cells were co-cultured overnight with dendritic cells expressing wildtype (wt) ATM or mutated forms of ATM (G2695V, G2695A, G2695C, or G2695S).

FIG. 9B contains a graph of interferon gamma (IFNγ) (pg/mL) in the supernatant after TCR-transduced T cells were co-cultured overnight with dendritic cells expressing wildtype (wt) ARID1A or mutated forms of ARID1A (1960fs).

FIG. 10 contains the amino acid sequences set forth in Table 3.

FIG. 11A contains the nucleotide sequence encoding the beta chain of TRBV9*01+TRAV14/DV4 chimeric TCR, with human beta variable region in larger font, CDR1, CDR2, CDR3 underlined, and mouse beta constant in italics (SEQ ID NO: 78).

FIG. 11B contains the nucleotide sequence encoding the alpha chain of TRBV9*01+TRAV14/DV4 chimeric TCR, with human alpha variable region in larger font, CDR1, CDR2, CDR3 underlined, and mouse alpha constant in italics (SEQ ID NO:79).

FIG. 12A contains the codon optimized nucleotide sequence encoding the beta chain of TRBV9*01+TRAV14/DV4 chimeric TCR, with human beta variable region in larger font, CDR1, CDR2, CDR3 underlined, and mouse beta constant in italics (SEQ ID NO:80).

FIG. 12B contains the codon optimized nucleotide sequence encoding the alpha chain of TRBV9*01+TRAV14/DV4 chimeric TCR, with human alpha variable region in larger font, CDR1, CDR2, CDR3 underlined, and mouse alpha constant in italics (SEQ ID NO:81).

FIG. 13A contains the nucleotide sequence encoding the beta chain of TRBV9*01+TRAV14/DV4 human TCR, with human beta variable region in larger font, CDR1, CDR2, CDR3 underlined, and human beta constant in italics (SEQ ID NO: 82).

FIG. 13B contains the nucleotide sequence encoding the alpha chain of TRBV9*01+TRAV14/DV4 human TCR, with human alpha variable region in larger font, CDR1, CDR2, CDR3 underlined, and human alpha constant in italics (SEQ ID NO: 83).

FIG. 14A contains the codon optimized nucleotide sequence encoding the beta chain of TRBV9*01+TRAV14/DV4 human TCR, with human beta variable region in larger font, CDR1, CDR2, CDR3 underlined, and human beta constant in italics (SEQ ID NO:84).

FIG. 14B contains the codon optimized nucleotide sequence encoding the alpha chain of TRBV9*01+TRAV14/DV4 human TCR, with human alpha variable region in larger font, CDR1, CDR2, CDR3 underlined, and human alpha constant in italics (SEQ ID NO:85).

FIG. 15A contains the nucleotide sequence encoding the beta chain of TRBV25-1+TRAV20 chimeric TCR with human beta variable region in larger font, CDR1, CDR2, CDR3 underlined, and mouse beta constant (codon optimized) in italics (SEQ ID NO:86).

FIG. 15B contains the nucleotide sequence encoding the alpha chain of TRBV25-1+TRAV20 chimeric TCR, with the human variable region in larger font, CDR1, CDR2, CDR3 underlined, and mouse alpha constant (codon optimized) in italics (SEQ ID NO:87).

FIG. 16A contains the codon optimized nucleotide sequence encoding the beta chain of TRBV25-1+TRAV20 chimeric TCR, with human variable region in larger font, CDR1, CDR2, CDR3 underlined, and mouse beta constant in italics (SEQ ID NO: 88).

FIG. 16B contains the codon optimized nucleotide sequence encoding the alpha chain of TRBV25-1+TRAV20 chimeric TCR, with human variable region in larger font, CDR1, CDR2, CDR3 underlined, and mouse alpha constant in italics (SEQ ID NO:89).

FIG. 17A contains the nucleotide sequence encoding the beta chain of TRBV25-1+TRAV20 human TCR, with human variable region in larger font, CDR1, CDR2, CDR3 underlined, and human beta constant in italics (SEQ ID NO:90).

FIG. 17B contains the nucleotide sequence encoding the alpha chain of TRBV25-1+TRAV20 human TCR, with human variable region in larger font, CDR1, CDR2, CDR3 underlined, and human alpha constant in italics (SEQ ID NO:91).

FIG. 18A contains the codon-optimized nucleotide sequence encoding the beta chain of TRBV25-1+TRAV20 human TCR, with human variable region in larger font, CDR1, CDR2, CDR3 underlined, and human beta constant in italics (SEQ ID NO: 92).

FIG. 18B contains the codon-optimized nucleotide sequence encoding the alpha chain of TRBV25-1+TRAV20 human TCR, with the human variable region in larger font, CDR1, CDR2, CDR3 underlined, and human alpha constant in italics (SEQ ID NO:93).

### DETAILED DESCRIPTION

In one aspect, this document provides TCRs having affinity for an ATM peptide, e.g., a mutant ATM peptide (within the context of an MHC molecule presenting that ATM peptide). In another aspect, this document provides TCRs having affinity for an ARID1A peptide, e.g., a mutant ARID1A peptide (within the context of an MHC molecule presenting that ARID1A peptide). The amino acid sequence of human ATM is set forth in NCBI Reference Sequence: NP_000042.3 or UniProt KB-Q13315 (ATM_HUMAN), and the amino acid sequence of human ARID1A is set forth in NCBI Reference Sequence: NP_006006.3 or UniProtKB-O14497 (ARIIA_HUMAN). See also Gene ID 472 for the human ATM gene and Gene ID 8289 for the human ARID1A gene. Mutations in ATM and ARID1A are commonly found, for example, in ovarian, endometrial, kidney, pancreas, gastric, bladder, lung, breast, brain, liver, cervical, uterine, prostate, and hematopoietic malignancies.

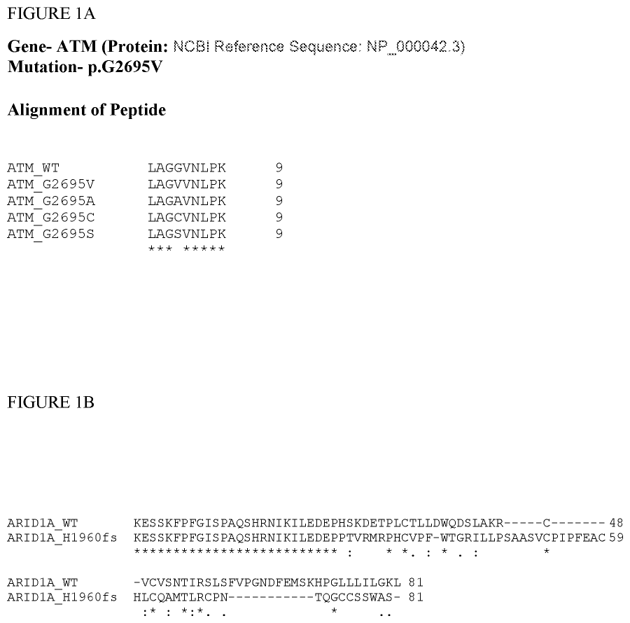

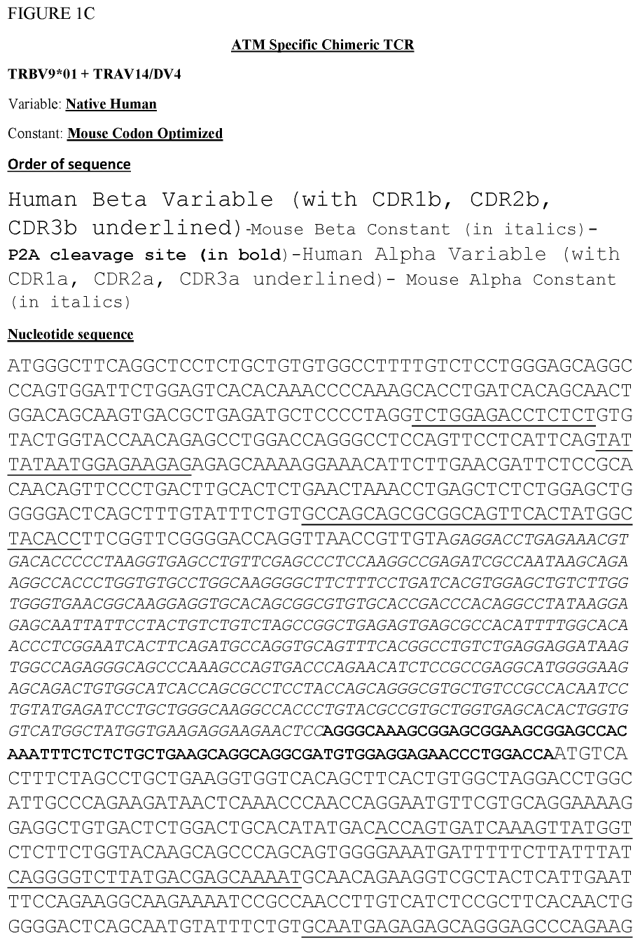

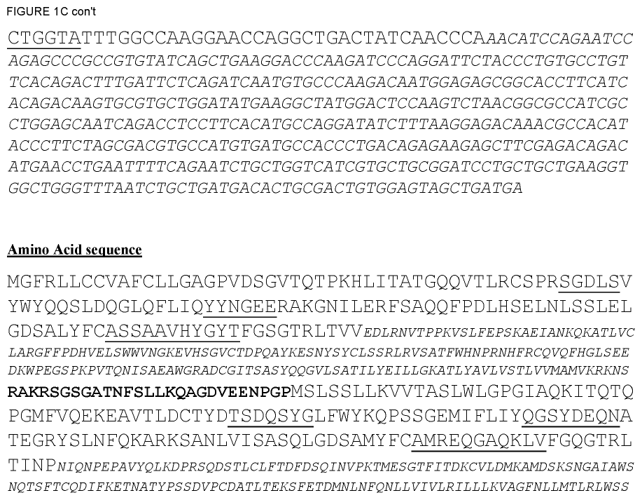

In some cases, a TCR provided herein can have specific affinity for a mutant ATM peptide (e.g., a mutant ATM peptide comprising a valine at the position corresponding to 2695 in the human ATM protein) within the context of an MHC molecule (e.g., HLA A*03:01) presenting that ATM peptide. For example, a TCR provided herein, and cells expressing such a TCR (e.g., immune cells), can have reactivity against a mutant ATM peptide comprising a valine, alanine, cysteine, or serine at the position corresponding to 2695 in the human ATM protein without having reactivity to the wild-type of ATM (e.g., having a glycine at the position corresponding to 2695 in the human ATM protein). See FIG. 1A for an alignment of mutant ATM peptides comprising a valine, alanine, cysteine, or serine at the position corresponding to 2695 in the human ATM protein (SEQ ID NOs: 61-64, respectively) and a wild-type ATM peptide (SEQ ID NO:65) having a glycine at the position corresponding to 2695 in the human ATM protein.

In some cases, a TCR provided herein can have specific affinity for a mutant ARID1A peptide (e.g., a mutant ARID1A peptide comprising a frameshift at the position corresponding to 1960 in the human ARID1A protein) within the context of an MHC molecule (e.g., HLA A*02:01) presenting that ARID1A peptide. For example, a TCR provided herein, and cells expressing such a TCR (e.g., immune cells), can have reactivity against a mutant ARID1A peptide comprising a frameshift at the position corresponding to 1960 in the human ARID1A protein without having reactivity to the wild-type of ARID1A (e.g., having a histidine at the position corresponding to 1960 in the human ARID1A protein). See FIG. 1B for an alignment of a mutant ARID1A peptide (SEQ ID NO:66) comprising a frameshift at the position corresponding to 1960 in the human ARID1A protein and a wild-type ARID1A peptide (SEQ ID NO:67) having a histidine at the position corresponding to 1960 in the human ARID1A protein.

A mutant ATM peptide or a mutant ARID1A peptide can be, for example, from 8 to 30 amino acids in length. Examples of mutant ATM peptides that can be recognized by a TCR provided herein are set forth in Table 1. Examples of mutant ARID1A peptides that can be recognized by a TCR provided herein are set forth in 5 Table 2.

TABLE 1
Exemplary mutant ATM peptides

| Sequence | SEQ ID NO: |
|---|---|
| LAGVVNLPK | 61 |
| LAGAVNLPK | 62 |
| LAGCVNLPK | 63 |
| LAGSVNLPK | 64 |

TABLE 2
Exemplary mutant ARID1A peptide

| | SEQ ID |
|---|---|
| Sequence | NO: |
| ILEDEPPTVRMRPHC | 68 |
| KESSKFPFGISPAQSHRNIKILEDE | 66 |
| PPTVRMRPHCVPFWTGRILLPSAAS | |
| VCPIPFEACHLCQAMTLRCPNTQGC | |
| CSSWAS | |
| NIKILEDEPPTVRMRPHCVPFWTGR | 94 |
| ILL | |

In some cases, immune cells that include an exogenous TCR provided herein (e.g., a TCR having affinity for a mutant ATM peptide, a TCR having affinity for an ARID1A peptide, or both a TCR having affinity for a mutant ATM peptide and a TCR having affinity for an ARID1A peptide) can be used to treat mammals (e.g., humans having cancer) who harbor the same or similar ATM and/or ARID1A mutations. For example, immune cells that include an exogenous TCR provided herein can be used to treat a subject with a cancer such as ovarian, endometrial, kidney, pancreas, gastric, bladder, lung, breast, brain, liver, cervical, uterine, prostate, or a hematopoietic malignancy.

A TCR is a heterodimeric cell surface protein and is involved in mediating signal transduction. The extracellular portion of a native heterodimeric TCR includes two polypeptide chains, each of which has a membrane-proximal constant domain, and a membrane-distal variable domain. The variable domains contain highly polymorphic loops analogous to the complementarity determining regions (CDRs) of antibodies.

In general, the TCRs provided herein include an alpha chain polypeptide and a beta chain polypeptide, where the alpha chain and the beta chain each include three CDRs and a constant region. In some cases, the alpha chain polypeptide of a TCR having affinity for an ATM peptide (e.g., a mutant ATM peptide) can include a CDR1 having the amino acid sequence set forth in SEQ ID NO:10 (or a variant with one, two, or three amino acid modifications), a CDR2 having the amino acid sequence set forth in SEQ ID NO:11 (or a variant with one, two, or three amino acid modifications), and a CDR3 having the amino acid sequence set forth in SEQ ID NO: 12 (or a variant with one, two, or three amino acid modifications), and the beta chain polypeptide can include a CDR1 having the amino acid sequence set forth in SEQ ID NO:1 (or a variant with one, two, or three amino acid modifications), a CDR2 having the amino acid sequence set forth in SEQ ID NO:2 (or a variant with one, two, or three amino acid modifications), and a CDR3 having the amino acid sequence set forth in SEQ ID NO:3 (or a variant with one, two, or three amino acid modifications).

In some cases, the alpha chain polypeptide of a TCR having affinity for an ARID1A peptide (e.g., a mutant ARID1A peptide) can include a CDR1 having the amino acid sequence set forth in SEQ ID NO:32 (or a variant with one, two, or three amino acid modifications), a CDR2 having the amino acid sequence set forth in SEQ ID NO: 33 (or a variant with one, two, or three amino acid modifications), and a CDR3 having the amino acid sequence set forth in SEQ ID NO:34 (or a variant with one, two, or three amino acid modifications), and the beta chain polypeptide can include a CDR1 having the amino acid sequence set forth in SEQ ID NO:23 (or a variant with one, two, or three amino acid modifications), a CDR2 having the amino acid sequence set forth in SEQ ID NO:24 (or a variant with one, two, or three amino acid modifications), and a CDR3 having the amino acid sequence set forth in SEQ ID NO: 25 (or a variant with one, two, or three amino acid modifications).

Examples of amino acid modifications with respect to the amino acid sequence set forth in SEQ ID NOs: 1, 2, 3, 10, 11, 12, 23, 24, 25, 32, 33, and 34 include, without limitation, amino acid substitutions, amino acid deletions, and amino acid additions. In some cases, a variant of SEQ ID NOs: 3, 12, 25, or 34 can include no more than one amino acid modification. Amino acid modifications can be made, for example, to improve the binding and/or contact with the ATM or ARID1A peptide.

In some cases, an amino acid substitution that can be engineered into a TCR containing the sequence set forth in SEQ ID NO:1, 2, 3, 10, 11, 12, 23, 24, 25, 32, 33, or 34 can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution that can be engineered into a TCR containing the sequence set forth in SEQ ID NO:1, 2, 3, 10, 11, 12, 23, 24, 25, 32, 33, or 34 can be a non-conservative amino acid substitution. Non-conservative amino acid substitutions can be made by substituting one amino acid residue for another amino acid residue having a dis-similar side chain. Examples of non-conservative substitutions that can be used as described herein include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating amino acid sequence variants can include site-specific mutagenesis or random mutagenesis (e.g., by PCR) of the nucleic acid encoding the alpha and/or beta chain polypeptide. See, for example, Zoller, Curr. Opin. Biotechnol. 3:348-354 (1992). Both naturally occurring and non-naturally occurring amino acids (e.g., artificially-derivatized amino acids) can be used to generate amino acid sequence variants of the alpha and/or beta chain polypeptides provided herein.

In some cases, the constant region of an alpha chain polypeptide of a TCR provided herein having affinity for an ATM peptide can have an amino acid sequence having at least 80% identity to the amino acid sequence set forth in SEQ ID NO:17 or SEQ ID NO:21. For example, the constant region of an alpha chain polypeptide of a TCR provided herein can have an amino acid sequence having at least 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:17 or SEQ ID NO:21.

In some cases, the variable region of an alpha chain polypeptide of a TCR provided herein having affinity for an ATM peptide can have an amino acid sequence having at least 80% identity to the amino acid sequence set forth in SEQ ID NO:45 provided that the alpha chain includes the appropriate combination of CDRs (or variants of the CDRs) described herein. For example, the variable region of an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 45, provided that it includes (a) SEQ ID NO:10 (or SEQ ID NO:10 with no more than three, two, or one amino acid modifications), (b) SEQ ID NO:11 (or SEQ ID NO: 11 with no more than three, two, or one amino acid modifications), and (c) SEQ ID NO:12 (or SEQ ID NO:12 with no more than three, two, or one amino acid modifications).

In some cases, the variable region of an alpha chain polypeptide of a TCR provided herein can include the amino acid sequence set forth in SEQ ID NO:45 (or a variant of SEQ ID NO:45 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:45 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:45 can include no more than one, two, three, four, or five amino acid modifications.

In some cases, an alpha chain polypeptide of a TCR provided herein having affinity for an ATM peptide can have an amino acid sequence having at least 80% identity to the amino acid sequence set forth in SEQ ID NO:18 or SEQ ID NO:22, provided that the alpha chain includes the appropriate combination of CDRs (or variants of the CDRs) described herein. For example, an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:18, provided that it includes (a) SEQ ID NO:10 (or SEQ ID NO:10 with no more than three, two, or one amino acid modifications), (b) SEQ ID NO:11 (or SEQ ID NO:11 with no more than three, two, or one amino acid modifications), and (c) SEQ ID NO: 12 (or SEQ ID NO:12 with no more than three, two, or one amino acid modifications). In another example, an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:22, provided that it includes (a) SEQ ID NO: 10 (or SEQ ID NO:10 with no more than three, two, or one amino acid modifications), (b) SEQ ID NO:11 (or SEQ ID NO:11 with no more than three, two, or one amino acid modifications), and (c) SEQ ID NO:12 (or SEQ ID NO:12 with no more than three, two, or one amino acid modifications).

In some cases, an alpha chain polypeptide of a TCR provided herein can include the amino acid sequence set forth in SEQ ID NO: 18 (or a variant of SEQ ID NO: 18 with one, two, three, four, five, six, seven, eight, nine, or 10 amino acid modifications, provided that the alpha chain includes the appropriate combination of CDRs (or variants of the CDRs) described herein) or SEQ ID NO:22 (or a variant of SEQ ID NO:22 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO: 18 or SEQ ID NO:22 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:18 or SEQ ID NO:22 can include no more than one, two, three, four, or five amino acid modifications.

In some cases, the constant region of a beta chain polypeptide of a TCR provided herein having affinity for an ATM peptide can have an amino acid sequence having at least 80% identity to the amino acid sequence set forth in SEQ ID NO:8 or SEQ ID NO:19. For example, the constant region of a beta chain polypeptide of a TCR provided herein can have an amino acid sequence having at least 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:8 or SEQ ID NO: 19.

In some cases, the variable region of a beta chain polypeptide of a TCR provided herein having affinity for an ATM peptide can have an amino acid sequence having at least 80% identity to the amino acid sequence set forth in SEQ ID NO:47, provided that the beta chain includes the appropriate combination of CDRs (or variants of the CDRs) described herein. For example, the variable region of a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 47, provided that it includes (a) SEQ ID NO:1 (or SEQ ID NO: 1 with no more than three, two, or one amino acid modifications), (b) SEQ ID NO:2 (or SEQ ID NO: 2 with no more than three, two, or one amino acid modifications), and (c) SEQ ID NO: 3 (or SEQ ID NO:3 with no more than three, two, or one amino acid modifications).

In some cases, the variable region of a beta chain polypeptide of a TCR provided herein can include the amino acid sequence set forth in SEQ ID NO:47 (or a variant of SEQ ID NO:47 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:47 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:47 can include no more than one, two, three, four, or five amino acid modifications.

In some cases, a beta chain polypeptide of a TCR provided herein having affinity for an ATM peptide can have an amino acid sequence having at least 80% identity to the amino acid sequence set forth in SEQ ID NO:9 or SEQ ID NO:20, provided that the beta chain includes the appropriate combination of CDRs (or variants of the CDRs) described herein. For example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:9, provided that it includes (a) SEQ ID NO:1 (or SEQ ID NO:1 with no more than three, two, or one amino acid modifications), (b) SEQ ID NO:2 (or SEQ ID NO:2 with no more than three, two, or one amino acid modifications), and (c) SEQ ID NO:3 (or SEQ ID NO:3 with no more than three, two, or one amino acid modifications). In another example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:20, provided that it includes (a) SEQ ID NO:1 (or SEQ ID NO:1 with no more than three, two, or one amino acid modifications), (b) SEQ ID NO:2 (or SEQ ID NO: 2 with no more than three, two, or one amino acid modifications), and (c) SEQ ID NO: 3 (or SEQ ID NO:3 with no more than three, two, or one amino acid modifications).

In some cases, a beta chain polypeptide of a TCR provided herein can have an amino acid sequence having at least 80% identity to the amino acid sequence set forth the amino acid sequence set forth in SEQ ID NO:9 (or a variant of SEQ ID NO:9 with one, two, three, four, five, six, seven, eight, nine, or 10 amino acid modifications, provided that the beta chain includes the appropriate combination of CDRs (or variants of the CDRs) described herein) or SEQ ID NO:20 (or a variant of SEQ ID NO: 20 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:9 or SEQ ID NO:20 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO: 9 or SEQ ID NO:20 can include no more than one, two, three, four, or five amino acid modifications.

In some cases, the constant region of an alpha chain polypeptide of a TCR provided herein having affinity for an ARID1A peptide can have an amino acid sequence having at least 80% identity to the amino acid sequence set forth in SEQ ID NO: 39 or SEQ ID NO:43. For example, the constant region of an alpha chain polypeptide of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:39 or SEQ ID NO:43.

In some cases, the variable region of an alpha chain polypeptide of a TCR provided herein having affinity for an ARID1A peptide can have an amino acid sequence having at least 80% identity to the amino acid sequence set forth in SEQ ID NO: 46, provided that the alpha chain includes the appropriate combination of CDRs (or variants of the CDRs) described herein. For example, the variable region of an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:46, provided that it includes (a) SEQ ID NO:32 (or SEQ ID NO:32 with no more than three, two, or one amino acid modifications), (b) SEQ ID NO:33 (or SEQ ID NO:33 with no more than three, two, or one amino acid modifications), and (c) SEQ ID NO:34 (or SEQ ID NO:34 with no more than three, two, or one amino acid modifications).

In some cases, the variable region of an alpha chain polypeptide of a TCR provided herein can include the amino acid sequence set forth in SEQ ID NO:46 (or a variant of SEQ ID NO:46 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:46 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:46 can include no more than one, two, three, four, or five amino acid modifications.

In some cases, an alpha chain polypeptide of a TCR provided herein having affinity for an ARID1A peptide can have an amino acid sequence having at least 80% identity to the amino acid sequence set forth in SEQ ID NO:40 or SEQ ID NO:44, provided that the alpha chain includes the appropriate combination of CDRs (or variants of the CDRs) described herein. For example, an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:40, provided that it includes (a) SEQ ID NO:32 (or SEQ ID NO:32 with no more than three, two, or one amino acid modifications), (b) SEQ ID NO:33 (or SEQ ID NO:33 with no more than three, two, or one amino acid modifications), and (c) SEQ ID NO: 34 (or SEQ ID NO:34 with no more than three, two, or one amino acid modifications). In another example, an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:44, provided that it includes (a) SEQ ID NO: 32 (or SEQ ID NO:32 with no more than three, two, or one amino acid modifications), (b) SEQ ID NO:33 (or SEQ ID NO:33 with no more than three, two, or one amino acid modifications), and (c) SEQ ID NO:34 (or SEQ ID NO:34 with no more than three, two, or one amino acid modifications).

In some cases, an alpha chain polypeptide of a TCR provided herein having affinity for an ARID1A peptide can include the amino acid sequence set forth in SEQ ID NO: 40 (or a variant of SEQ ID NO:40 with one, two, three, four, five, six, seven, eight, nine, or 10 amino acid modifications, provided that the alpha chain includes the appropriate combination of CDRs (or variants of the CDRs) described herein) or SEQ ID NO: 44 (or a variant of SEQ ID NO:44 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:40 or SEQ ID NO:44 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:40 or SEQ ID NO:44 can include no more than one, two, three, four, or five amino acid modifications.

In some cases, the constant region of a beta chain polypeptide of a TCR provided herein having affinity for an ARID1A peptide can have an amino acid sequence having at least 80% identity to the amino acid sequence set forth in SEQ ID NO: 30 or SEQ ID NO:41. For example, the constant region of a beta chain polypeptide of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:30 or SEQ ID NO:41.

In some cases, the variable region of a beta chain polypeptide of a TCR provided herein having affinity for an ARID1A peptide can have an amino acid sequence having at least 80% identity to the amino acid sequence set forth in SEQ ID NO: 48, provided that the beta chain includes the appropriate combination of CDRs (or variants of the CDRs) described herein. For example, the variable region of a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO: 48, provided that it includes (a) SEQ ID NO:23 (or SEQ ID NO:23 with no more than three, two, or one amino acid modifications), (b) SEQ ID NO:24 (or SEQ ID NO: 24 with no more than three, two, or one amino acid modifications), and (c) SEQ ID NO:25 (or SEQ ID NO:25 with no more than three, two, or one amino acid modifications).

In some cases, the variable region of a beta chain polypeptide of a TCR provided herein can include the amino acid sequence set forth in SEQ ID NO:48 (or a variant of SEQ ID NO:48 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:48 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:48 can include no more than one, two, three, four, or five amino acid modifications.

In some cases, a beta chain polypeptide of a TCR provided herein having affinity for an ARID1A peptide can have an amino acid sequence having at least 80% identity to the amino acid sequence set forth in SEQ ID NO:31 or SEQ ID NO:42, provided that the beta chain includes the appropriate combination of CDRs (or variants of the CDRs) described herein. For example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:31, provided that it includes (a) SEQ ID NO:23 (or SEQ ID NO:23 with no more than three, two, or one amino acid modifications), (b) SEQ ID NO:24 (or SEQ ID NO:24 with no more than three, two, or one amino acid modifications), and (c) SEQ ID NO:25 (or SEQ ID NO: 25 with no more than three, two, or one amino acid modifications). In another example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:42, provided that it includes (a) SEQ ID NO:23 (or SEQ ID NO: 23 with no more than three, two, or one amino acid modifications), (b) SEQ ID NO: 24 (or SEQ ID NO:24 with no more than three, two, or one amino acid modifications), and (c) SEQ ID NO:25 (or SEQ ID NO:25 with no more than three, two, or one amino acid modifications).

In some cases, a beta chain polypeptide of a TCR provided herein having affinity for an ARID1A peptide can have an amino acid sequence having at least 80% identity to the amino acid sequence set forth the amino acid sequence set forth in SEQ ID NO: 31 (or a variant of SEQ ID NO:31 with one, two, three, four, five, six, seven, eight, nine, or 10 amino acid modifications, provided that the beta chain includes the appropriate combination of CDRs (or variants of the CDRs) described herein) or SEQ ID NO: 42 (or a variant of SEQ ID NO:42 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:31 or SEQ ID NO:42 can be conservative amino acid substitutions or non-conservative amino acid substitutions as discussed above for SEQ ID NOs: 1, 2, 3, 10, 11, 12, 23, 24, 25, 32, 33, and 34. In some cases, a variant of SEQ ID NO:31 or SEQ ID NO:42 can include no more than one, two, three, four, or five amino acid modifications.

In some cases, a TCR provided herein can include no more than four (e.g., four, three, two, or one) amino acid modifications (e.g., amino acid substitutions, amino acid deletions, or amino acid additions) in one, two, three, or all four of the framework regions (the regions between the CDRs). For example, a TCR provided herein that has affinity for a ATM peptide can include no more than four (e.g., four, three, two, or one) amino acid modifications in one, two, three, or all four of the alpha chain framework regions set forth in SEQ ID NOs: 13, 14, 15, and 16 and/or the beta chain framework regions set forth in SEQ ID NOs: 4, 5, 6, and 7. For example, a TCR provided herein that has affinity for a ARID1A peptide can include no more than four (e.g., four, three, two, or one) amino acid modifications in one, two, three, or all four of the alpha chain framework regions set forth in SEQ ID NOs: 35, 36, 37, and 38 and/or the beta chain framework regions set forth in SEQ ID NOs: 26, 27, 28, and 29. In some cases, a TCR provided herein can include no more than four (e.g., four, three, two, or one) amino acid modifications (e.g., amino acid substitutions, amino acid deletions, or amino acid additions) in one, two, three, or all four of the framework regions set forth in SEQ ID NOs: 4, 5, 6, 7, 13, 14, 15, and 16 and (i) one, two, or three amino acid modifications in one, two, or all three CDRs of the alpha chain variable region having the amino acid sequences set forth in SEQ ID NOs: 10, 11, and 12 and/or (ii) one, two, or three amino acid modifications in one, two, or all three CDRs of the beta chain variable region having the amino acid sequences set forth in SEQ ID NOs: 1, 2, and 3. In some cases, a TCR provided herein can include no more than four (e.g., four, three, two, or one) amino acid modifications (e.g., amino acid substitutions, amino acid deletions, or amino acid additions) in one, two, three, or all four of the framework regions set forth in SEQ ID NOs: 26, 27, 28, 29, 35, 36, 37, and 38 and (i) one, two, or three amino acid modifications in one, two, or all three CDRs of the alpha chain variable region having the amino acid sequences set forth in SEQ ID NOs: 32, 33, and 34 and/or (ii) one, two, or three amino acid modifications in one, two, or all three CDRs of the beta chain variable region having the amino acid sequences set forth in SEQ ID NOs: 23, 24, and 25.

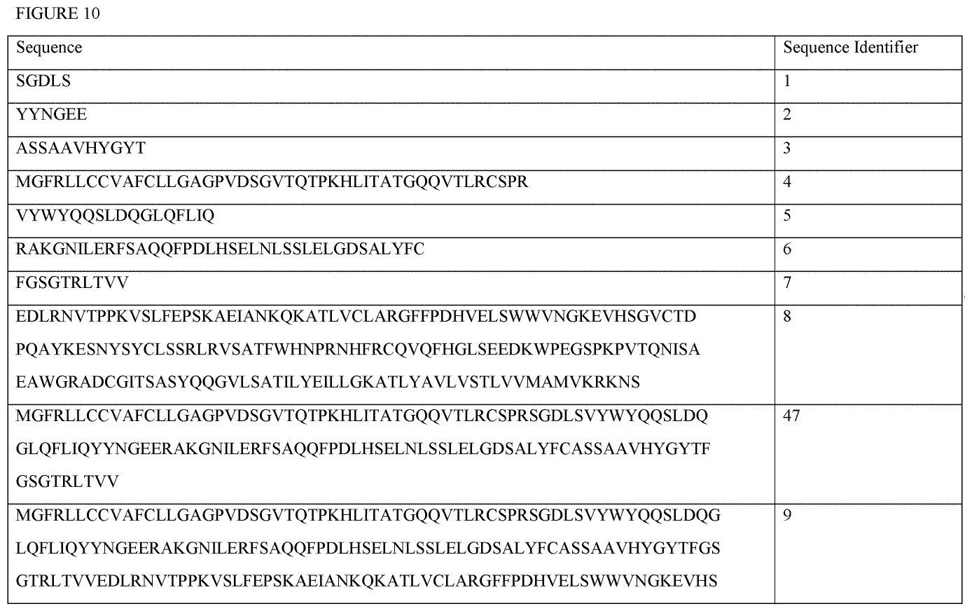

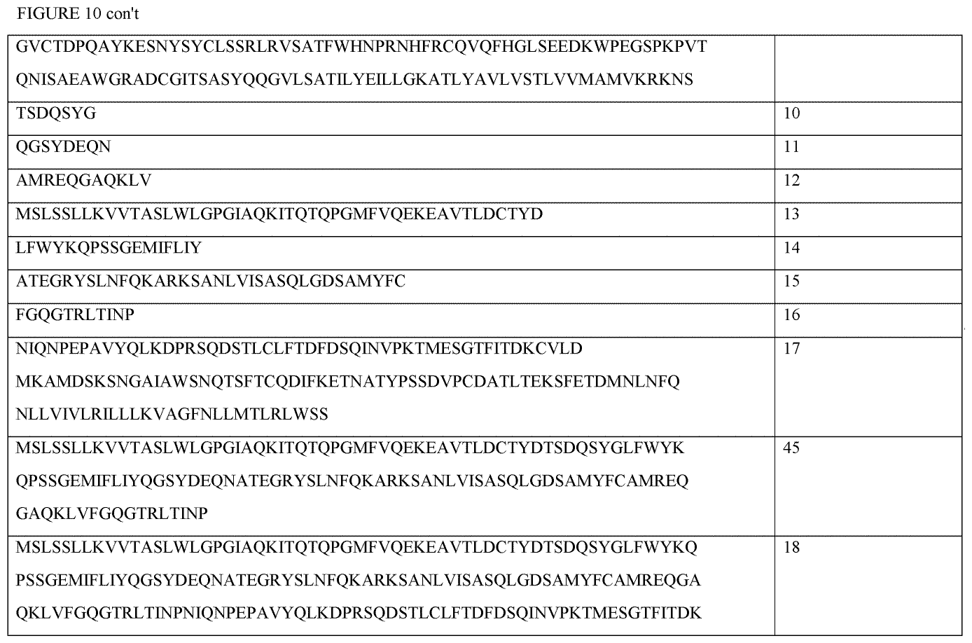

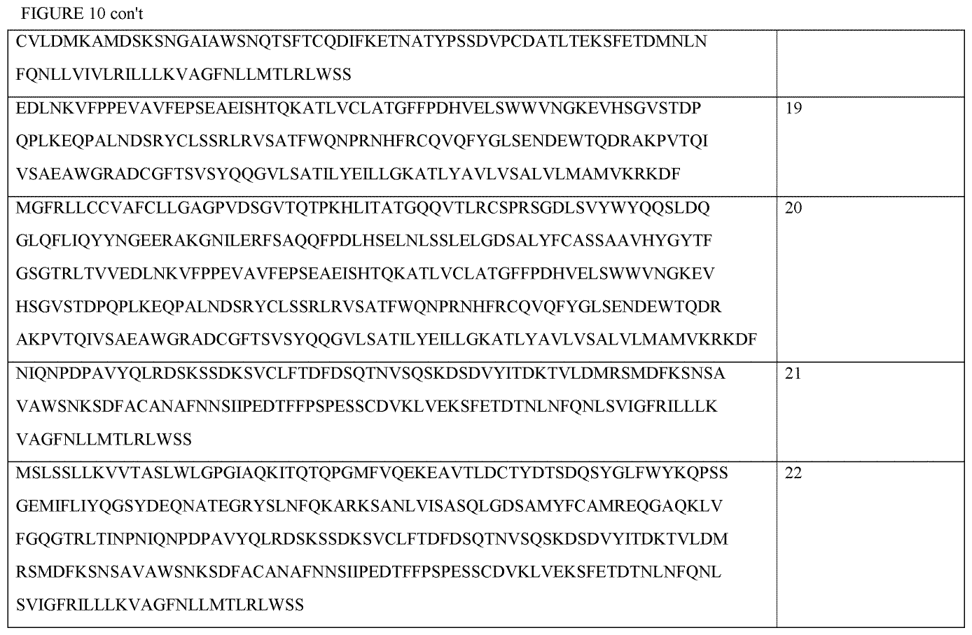

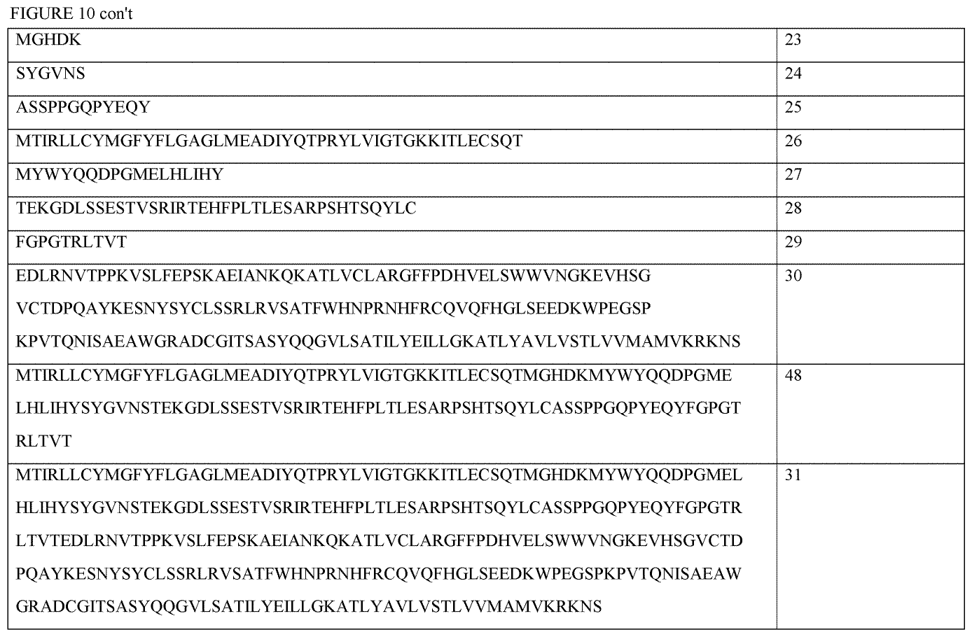

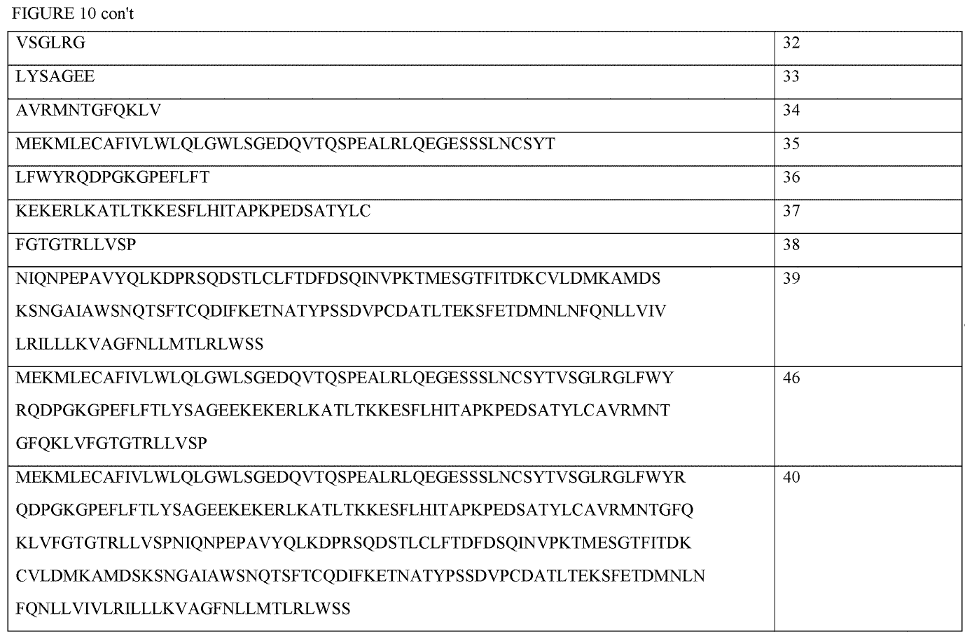

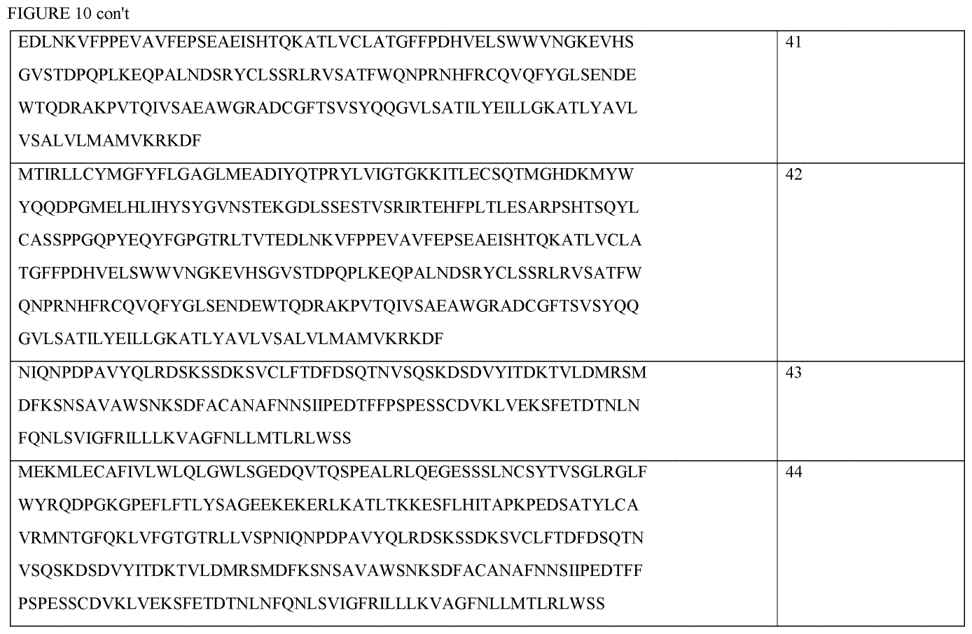

In some cases, a TCR provided herein can have the sequences as set forth in Table 3 (see FIG. 10 for the sequences).

TABLE 3
Exemplary TCRs.

| | | SEQ ID | | | | | SEQ ID | | | |
|---|---|---|---|---|---|---|---|---|---|---|
| | SEQ ID | NO of | | | | SEQ ID | NO of | SEQ ID of | | |
| | NO of | beta | SEQ ID of | SEQ ID | SEQ ID | NOs of | alpha | Constant | SEQ ID | SEQ ID |
| | beta | chain | constant | NO of | NO of | alpha | chain | region | NO of | NO of |
| | chain | Framework | region in | variable | beta | chain | Framework | in alpha | variable | alpha |
| TCR | CDRs | regions | beta chain | region | chain | CDRs | regions | chain | region | chain |
| TRBV9*01 + | 1, 2, 3 | 4, 5, 6, 7 | 8 | 47 | 9 | 10, 11, 12 | 13, 14, 15, 16 | 17 | 45 | 18 |
| TRAV14/DV4 | | | | | | | | | | |
| chimeric TCR | | | | | | | | | | |
| TRBV9*01 + | 1, 2, 3 | 4, 5, 6, 7 | 19 | 47 | 20 | 10, 11, 12 | 13, 14, 15, 16 | 21 | 45 | 22 |
| TRAV14/DV4 | | | | | | | | | | |
| Human TCR | | | | | | | | | | |
| TRBV25-1 + | 23, 24, 25 | 26, 27, 28, 29 | 30 | 48 | 31 | 32, 33, 34 | 35, 36, 37, 38 | 39 | 46 | 40 |
| TRAV20 | | | | | | | | | | |
| Chimeric | | | | | | | | | | |
| TCR | | | | | | | | | | |
| TRBV25-1 + | 23, 24, 25 | 26, 27, 28, 29 | 41 | 48 | 42 | 32, 33, 34 | 35, 36, 37, 38 | 43 | 46 | 44 |
| TRAV20 | | | | | | | | | | |
| Human TCR | | | | | | | | | | |

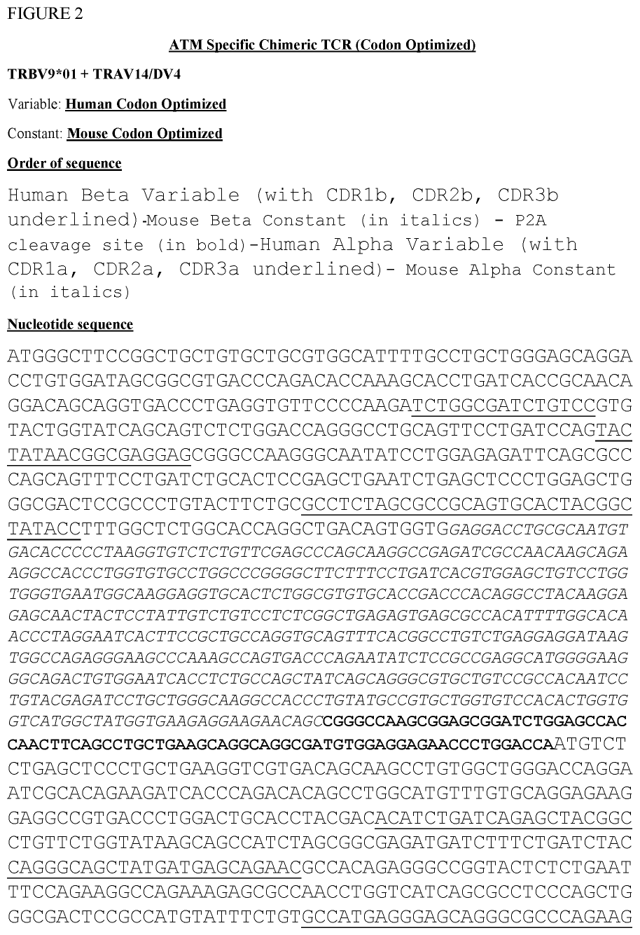

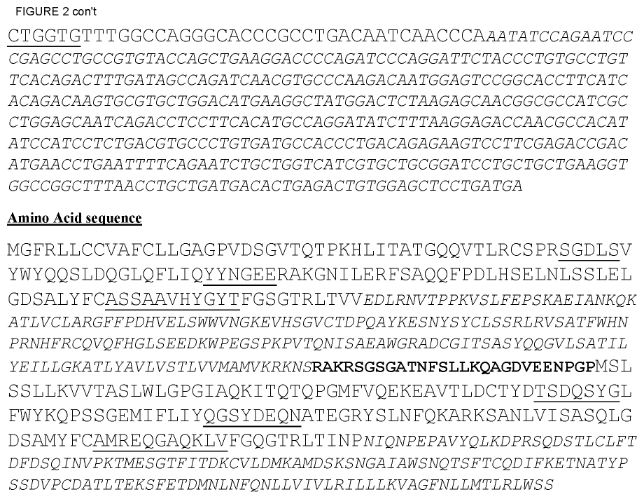

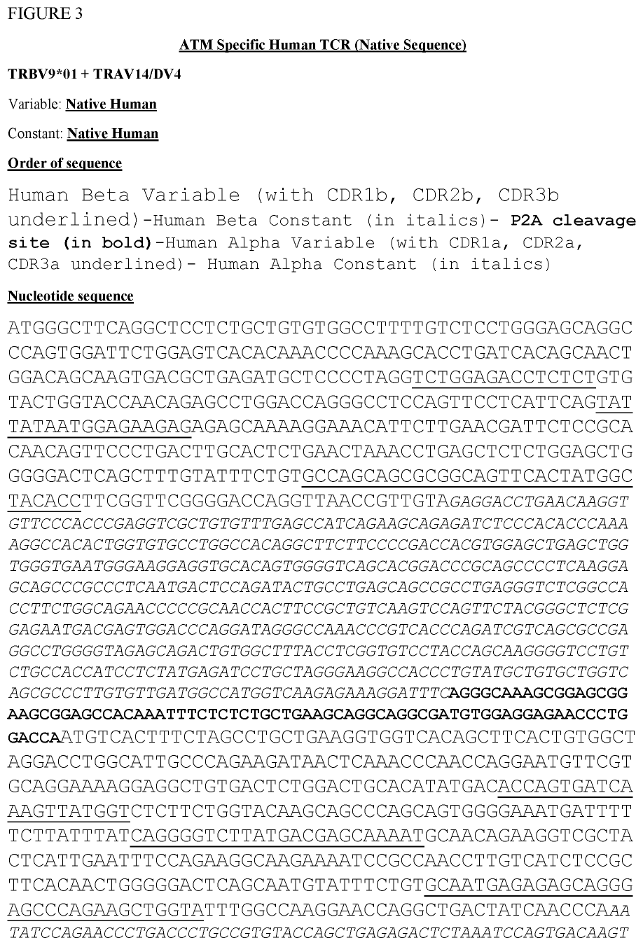

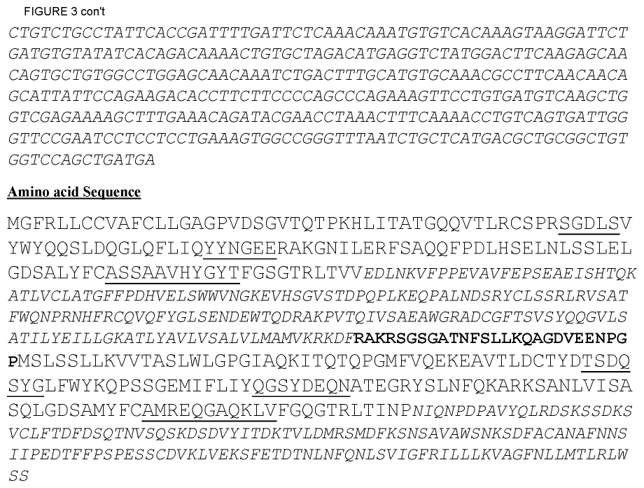

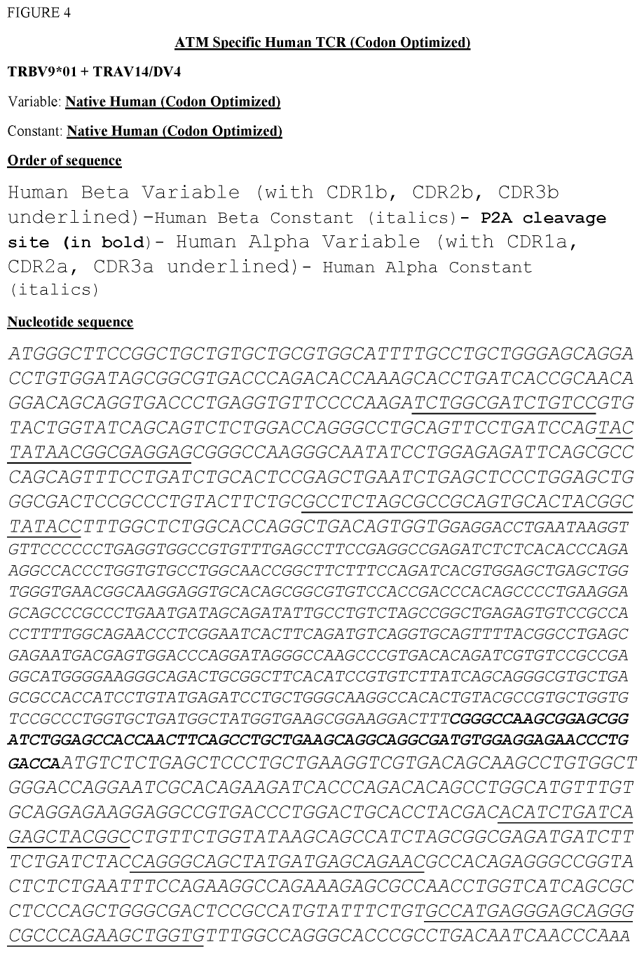

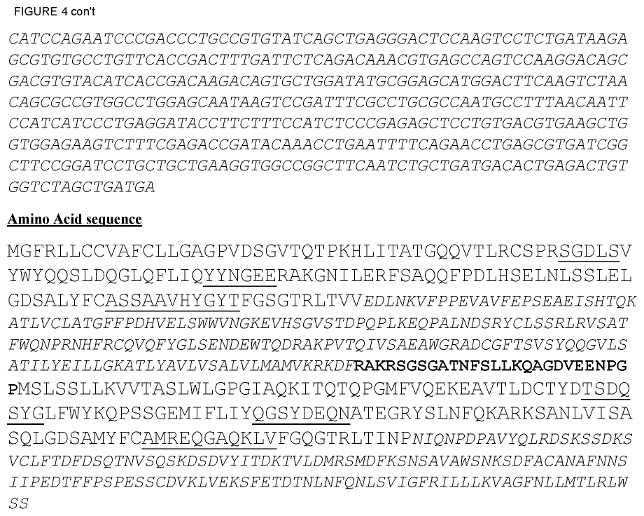

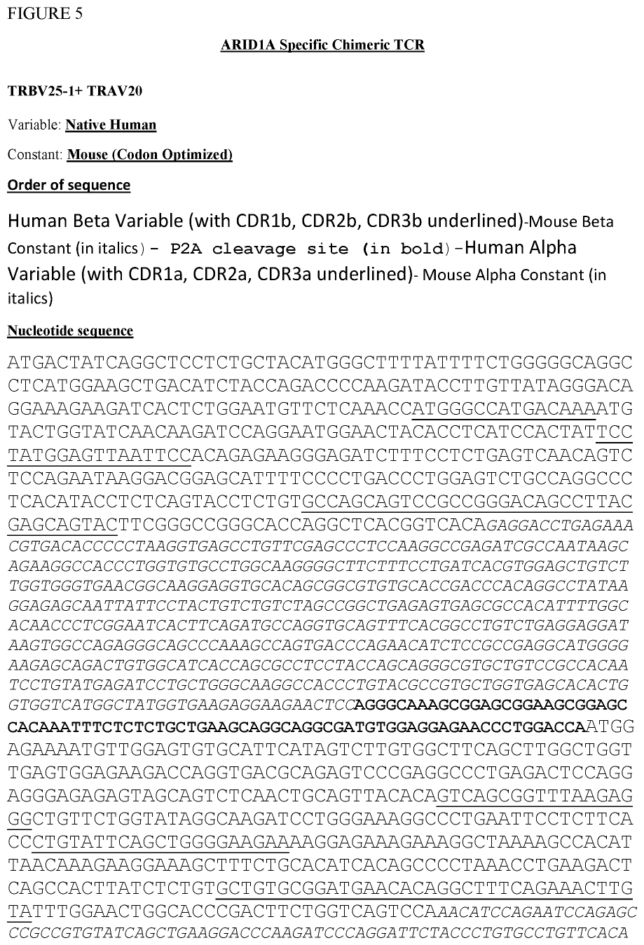

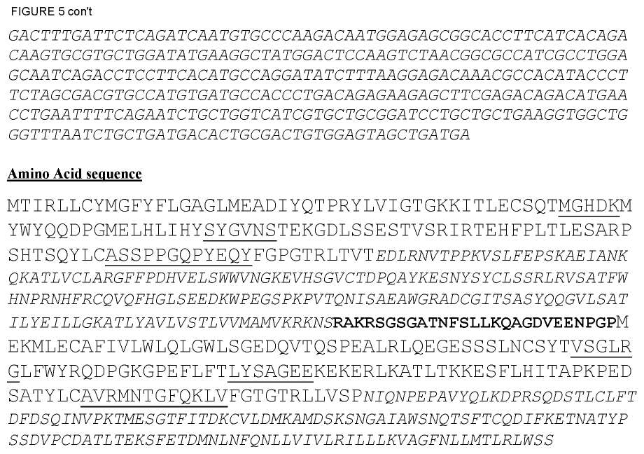

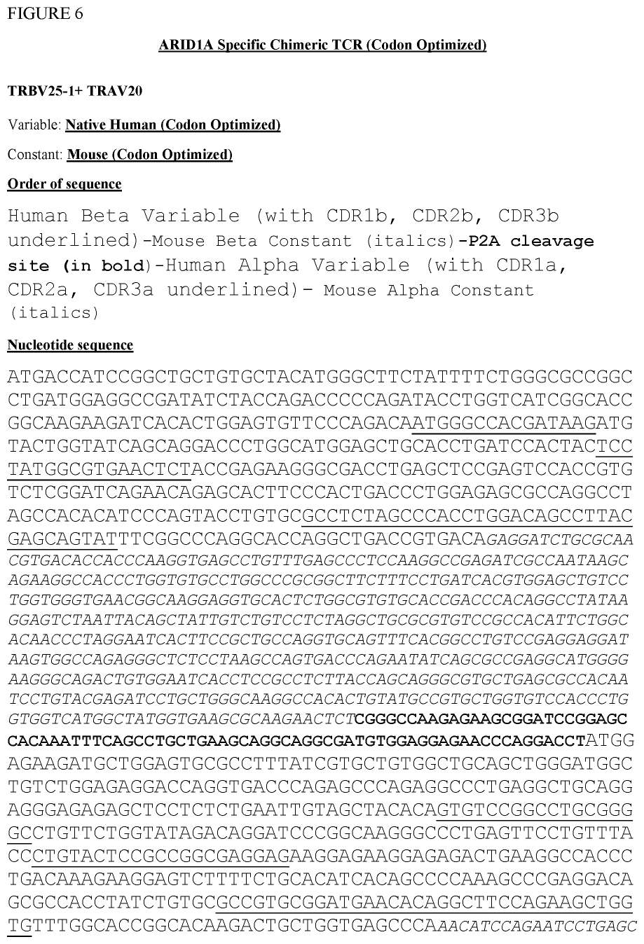

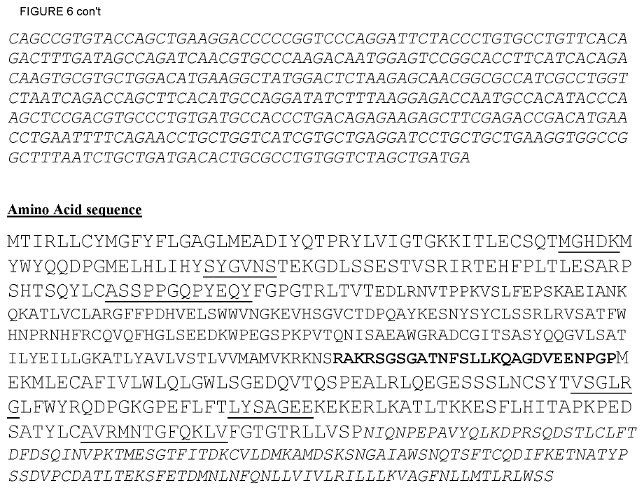

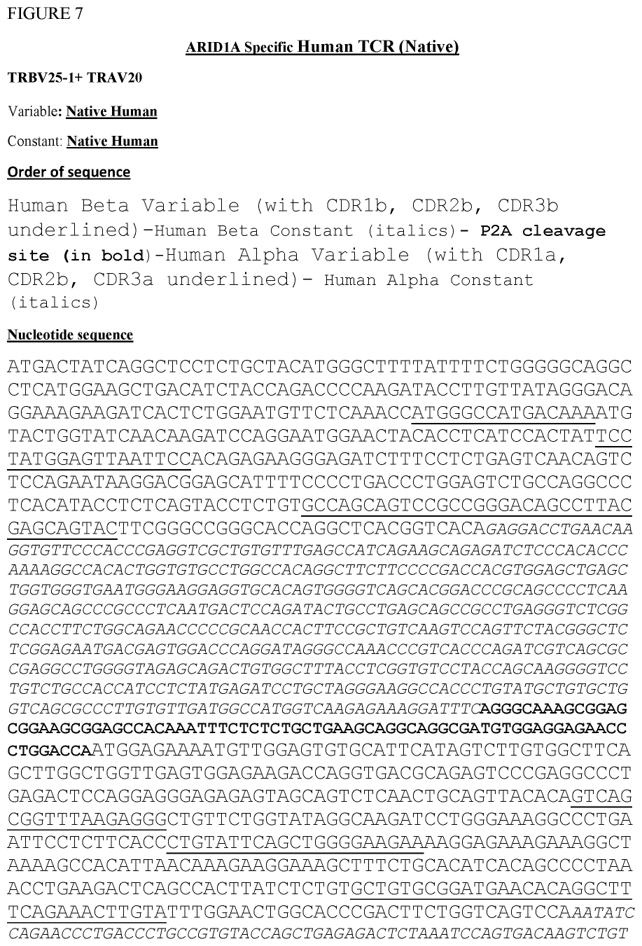

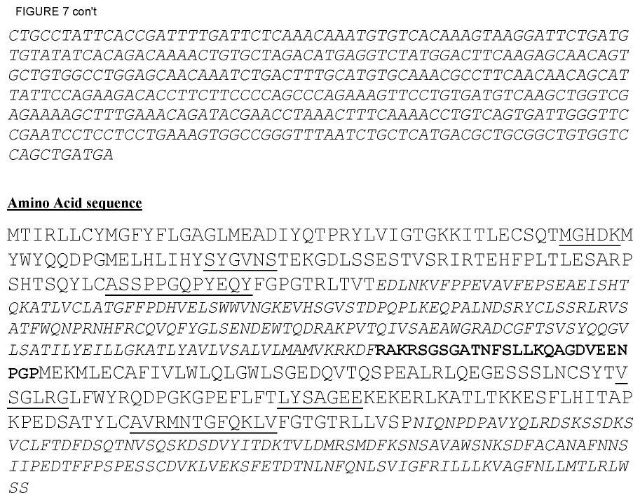

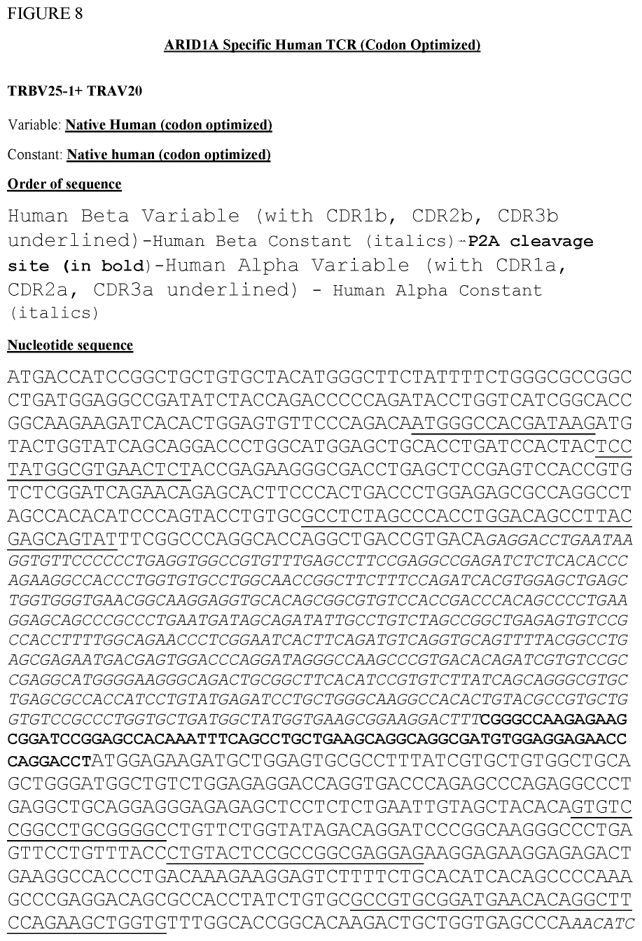

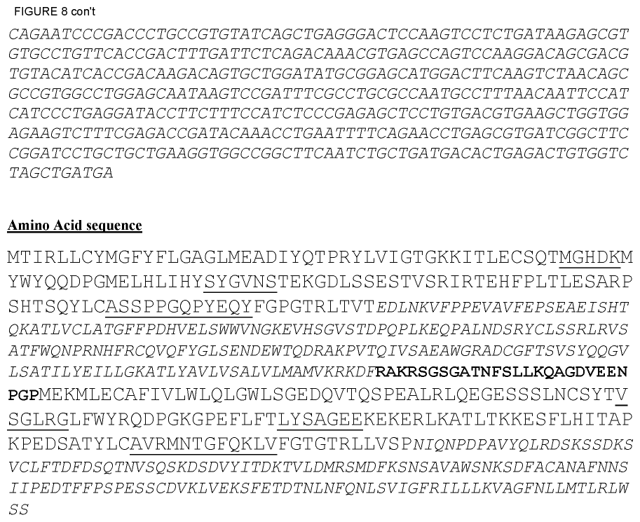

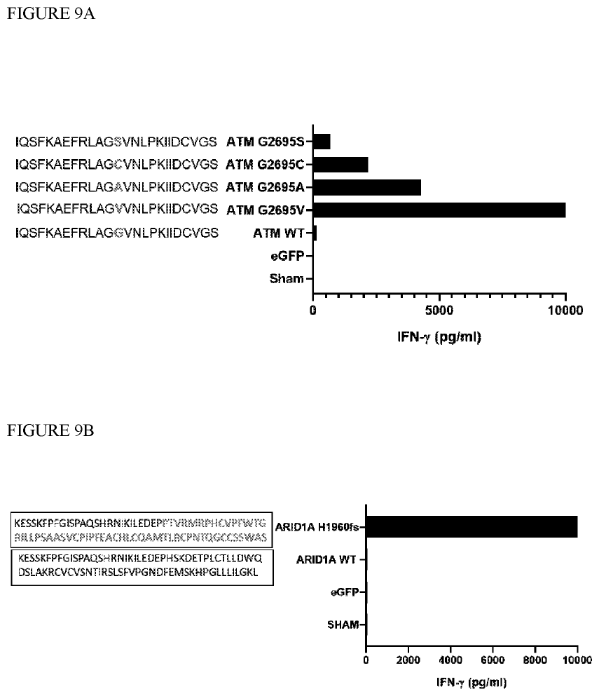

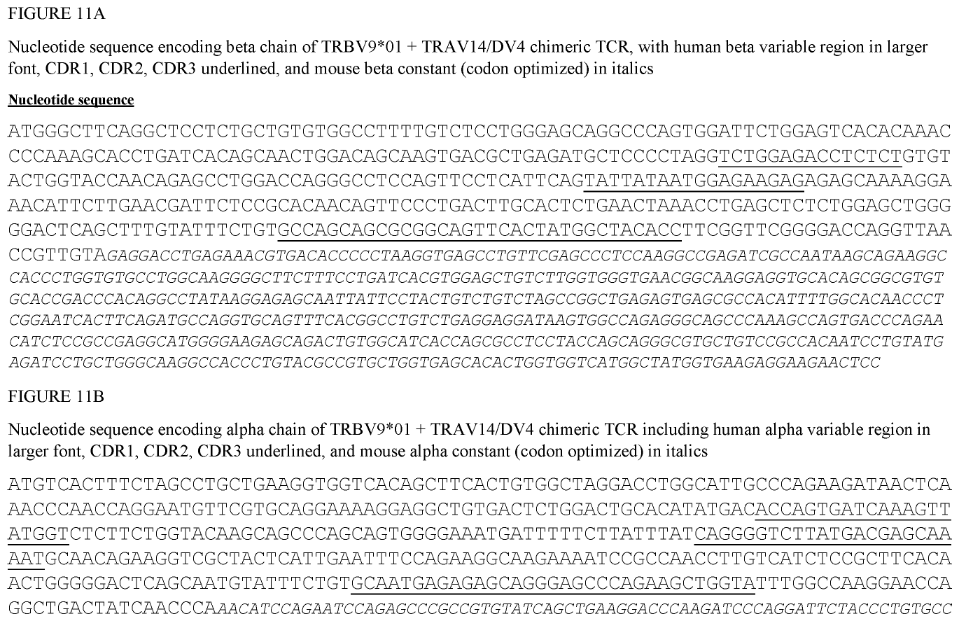

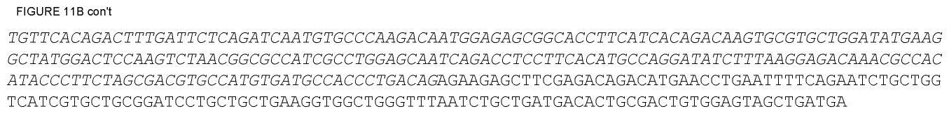

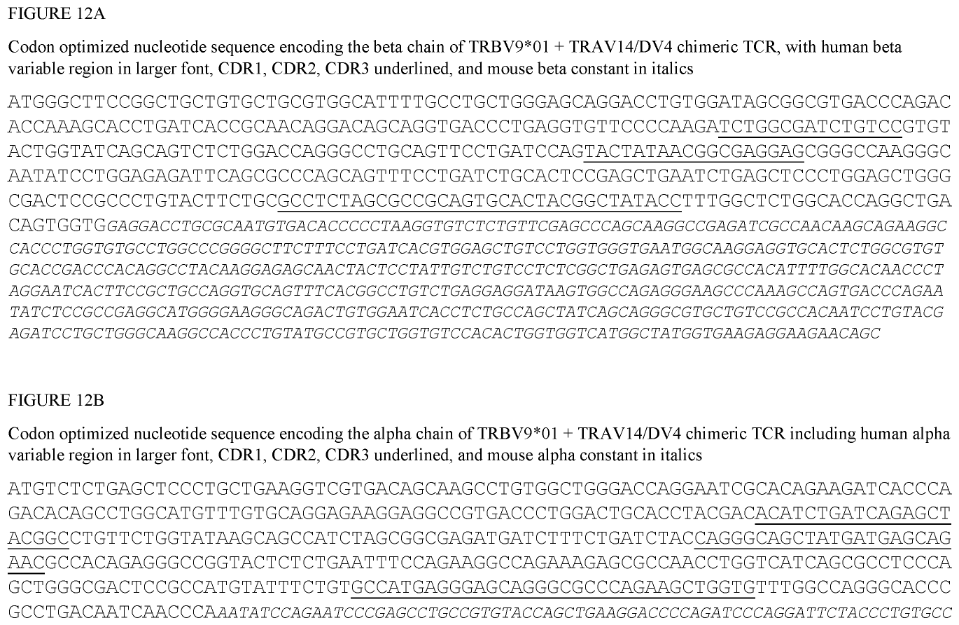

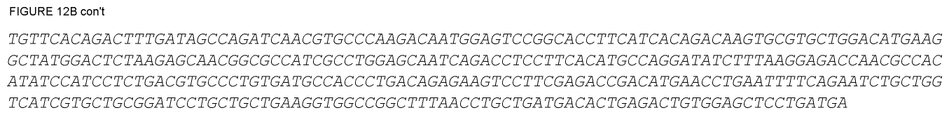

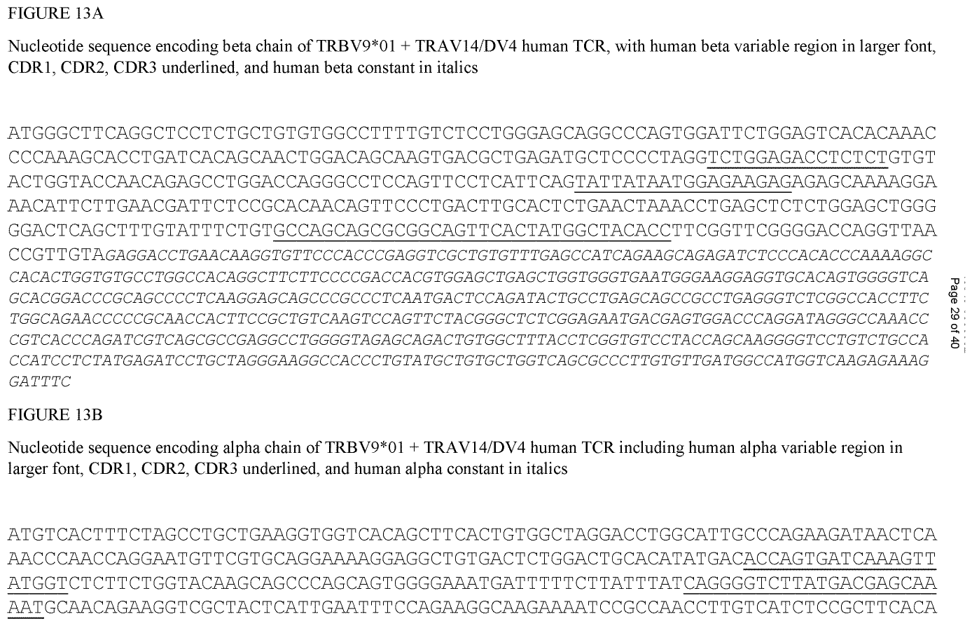

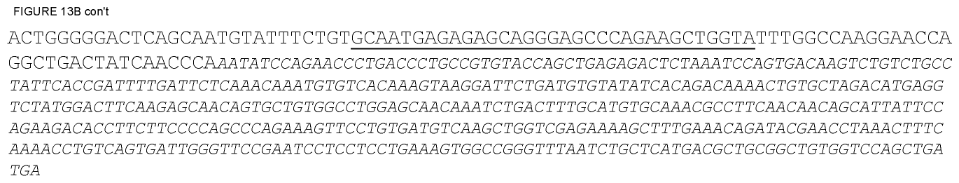

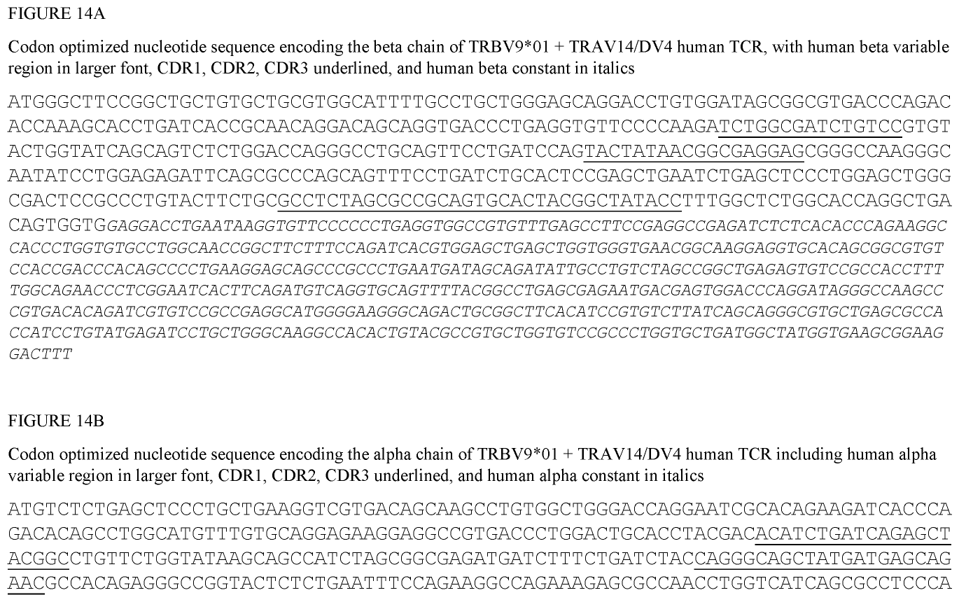

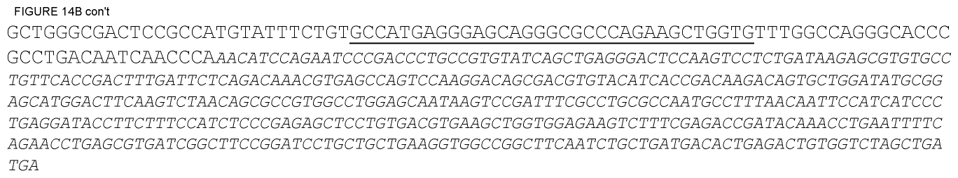

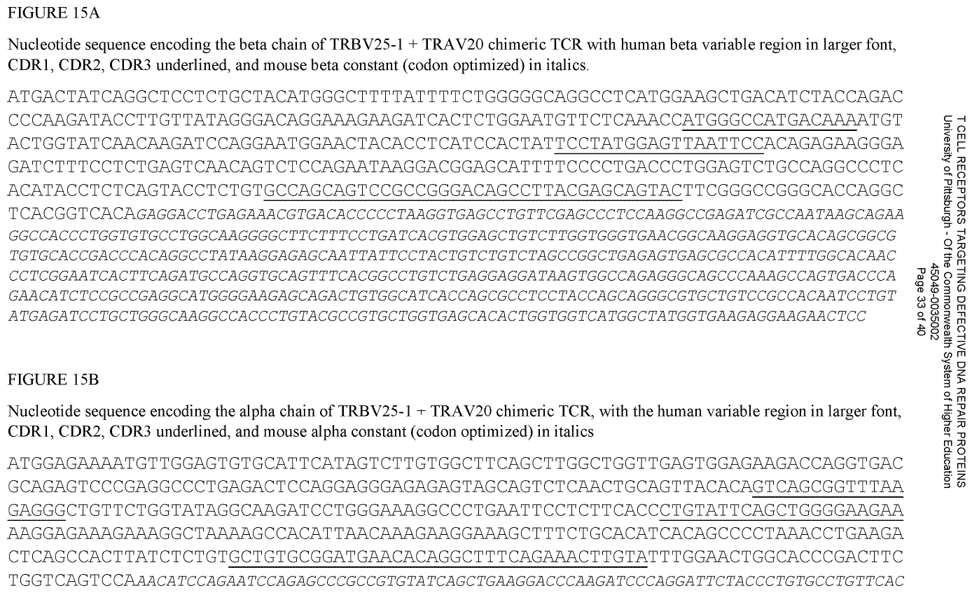

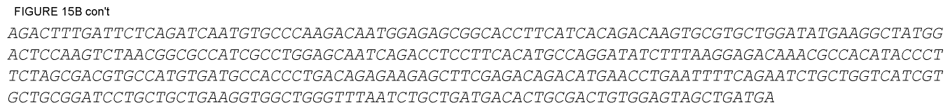

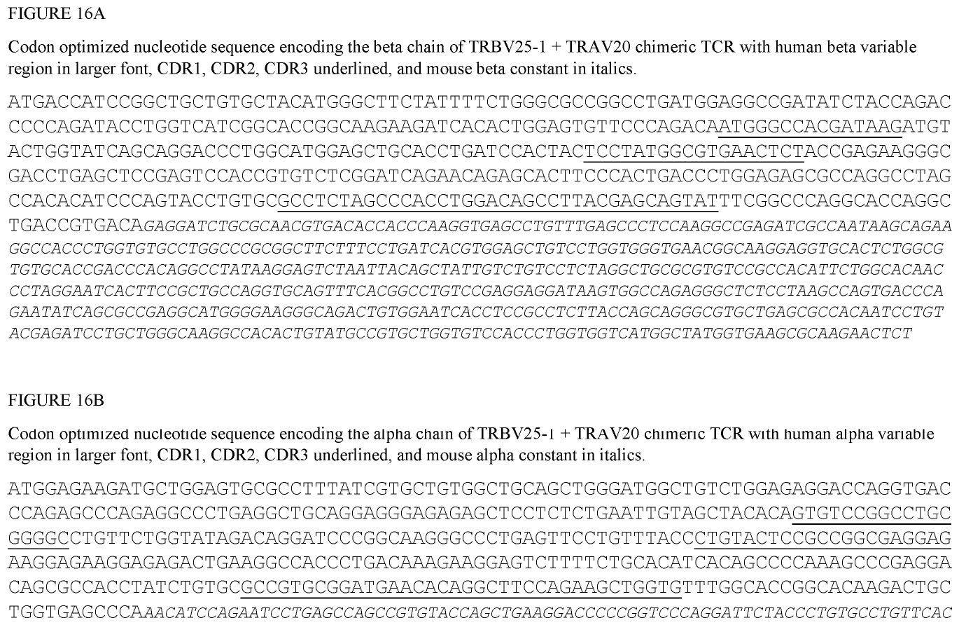

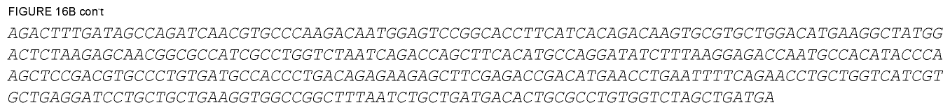

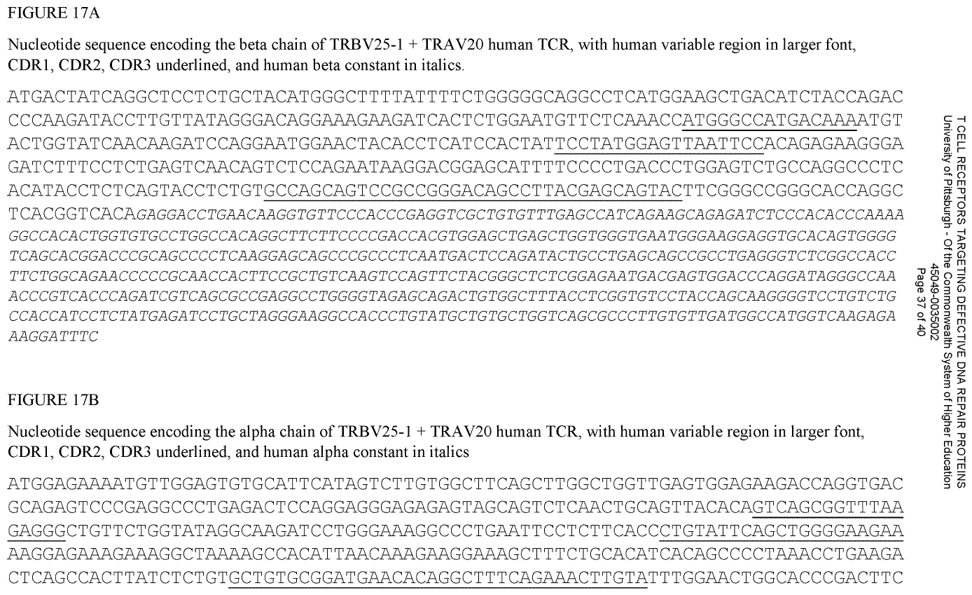

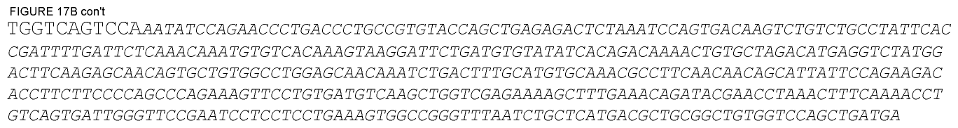

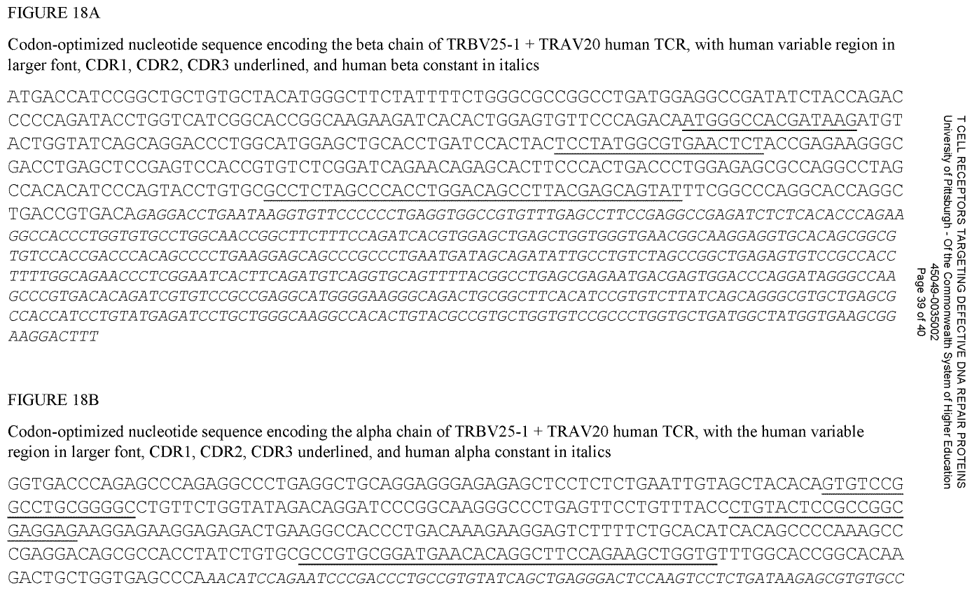

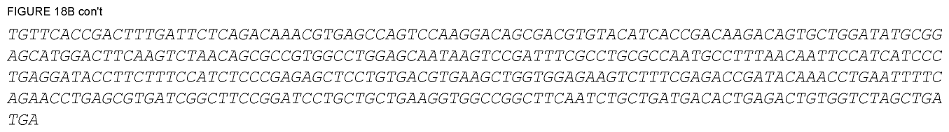

This document also provides isolated nucleic acids that encode an alpha chain polypeptide of a TCR provided herein, a beta chain polypeptide of a TCR provided herein, or both an alpha chain polypeptide of a TCR provided herein and a beta chain polypeptide of a TCR provided herein. See, for example, FIG. 1C, FIG. 3, FIG. 5, FIG. 9, FIG. 11A, FIG. 11B, FIG. 13A, FIG. 13B, FIG. 15A, FIG. 15B, FIG. 17A, or FIG. 17B. In some cases, the nucleic acid sequence can be codon optimized for expression in a host cell (e.g., a human host cell). See, for example, FIG. 2, FIG. 4, FIG. 6, FIG. 8, FIG. 12A, FIG. 12B, FIG. 14A, FIG. 14B, FIG. 16A, FIG. 16B, FIG. 18A, or FIG. 18B. In addition, this document provides vectors containing one or more of such nucleic acids. The nucleic acids provided herein can be single stranded and double stranded nucleic acids of any appropriate type (e.g., DNA, RNA, or DNA/RNA hybrids). The nucleic acids provided herein can be used therapeutically or can be used in methods for producing a TCR.

In some cases, a nucleic acid provided herein includes a nucleic acid sequence that encodes an alpha chain of a TCR and a nucleic acid sequence that encodes a beta chain of a TCR. The nucleic acid also can include a nucleic acid sequence encoding a linker. The nucleic acid sequence encoding the linker can be between the nucleic acid encoding an alpha chain and the nucleic acid encoding the beta chain to allow the alpha and beta chains to be encoded by the same contiguous nucleic acid sequence. For example, the nucleic acid sequence encoding the linker can be 3′ of the nucleic acid sequence encoding the alpha chain and 5′ of the nucleic acid sequence encoding the beta chain or it can be 3′ of the nucleic acid sequence encoding the beta chain and 5′ of the nucleic acid sequence encoding the alpha chain.

In some cases, the linker can be a self-cleaving peptide (e.g., a 2A peptide) such that during translation of the transcripts, the growing polypeptide can be cleaved at the 2A peptide with translation continuing through to the next chain. When designing a vector to express the alpha and beta chains as a multicistronic unit, the nucleic acid encoding the alpha and beta chains and the self-cleaving peptide (e.g., a 2A peptide) can be designed such that they are in translational frame with each other. Examples of 2A peptides that can be used as described herein include, without limitation, a 2A peptide of foot-and-mouth disease virus (FMDV), a 2A peptide of equine rhinitis A virus (ERAVO), a 2A peptide of Thosea asigna virus (TaV), or a 2A peptide of porcine teschovirus-1 (PTV-1). 2A peptides derived from FMDV, ERAV, PTV-1, and TaV are referred to herein as “F2A,” “E2A,” “P2A,” and “T2A,” respectively. Table 4 provides exemplary amino acid sequences of 2A peptides.

TABLE 4
Exemplary 2A peptides that can be
used as described herein.

| | | SEQ |
|---|---|---|
| | | ID |
| Type | Peptide Sequence | NO: |
| P2A | GSGATNFSLLKQAGDVEENPGP | 69 |
| | or | |
| | RAKRSGSGATNFSLLKQAGDVE | 95 |
| | ENPGP | |
| T2A | GSGEGRGSLLTCGDVEENPGP | 70 |
| E2A | GSGQCTNYALLKLAGDVESNP | 71 |
| | GP | |
| F2A | GSGVKQTLNFDLLKLAGDV | 72 |
| | ESNPGP | |

In some cases, an Internal Ribosome Entry Site (IRES) can be used in place of (or in addition to) a self-cleaving peptide. Examples of IRES sequences that can be used as described herein include, without limitation, an Encephalomyocrditis virus (EMCV) IRES (e.g., IRES2), a Hepatitis C virus (HCV) IRES, a Picorna virus IRES, and a Pestivirus IRES.

In some cases, a linker can include a furin cleavage site. Furin is a ubiquitously expressed protease that resides in the trans-golgi and processes protein precursors before their secretion. Furin cleaves at the COOH terminus of its consensus recognition sequence. Examples of furin consensus recognition sequences (or “furin cleavage sites”) that can be used as described herein include, without limitation, Arg-X-Lys-Arg (SEQ ID NO:73) or Arg-X-Arg-Arg (SEQ ID NO:74), (Lys/Arg)-Arg-X-(Lys/Arg)-Arg (SEQ ID NO:75) and Arg-X-X-Arg (SEQ ID NO: 76), such as an Arg-Gln-Lys-Arg (SEQ ID NO:77), where X is any naturally occurring amino acid.

A vector that includes one or more nucleic acids that encode an alpha chain and/or beta chain of a TCR provided herein can be a nucleic acid vector (e.g., naked DNA or plasmid vector) or a viral vector. Examples of viral vectors that can be designed to include one or more nucleic acids encoding an alpha chain and/or beta chain of a TCR provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), or poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18 (2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids encoding an alpha chain and/or beta chain of a TCR provided herein. Any appropriate nucleic acid or viral vector construction methods can be used to make a nucleic acid vector that includes one or more nucleic acids encoding an alpha chain and/or beta chain of a TCR provided herein (see, e.g., Green and Sambrook, Molecular Cloning: A Laboratory Manual, 4th edition, Cold Spring Harbor Laboratory, NY (2012); and Ausubel et al., Current Protocols in Molecular Biology, Green Publishing Associates and John Wiley & Sons, New York, N.Y. (1987-2008)).

A vector provided herein (e.g., a nucleic acid or viral vector provided herein) can include any appropriate promoter and/or other regulatory sequences (e.g., transcription and translation initiation and termination codons) operably linked the nucleic acid encoding a polypeptide (e.g., an alpha chain and/or beta chain of a TCR provided herein). In some cases, a promoter used to drive expression of an alpha chain and/or beta chain of a TCR provided herein can be a constitutive or regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, and AAV promoters. In some embodiments, the promoter can be a viral 5′ long terminal repeat (LTR) or 3′ LTR.

In some cases, a vector includes a separate promoter to drive expression of each chain instead of using a linker between the chains. In these cases, one promoter sequence can drive expression of an alpha chain, and a separate promoter sequence can drive expression of a beta chain. These two promoter sequences can be the same or different.

This document also provides cells (e.g., host cells or isolated cells) that include at least one nucleic acid provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein). Such cells (e.g., host cells or isolated cells) that can be designed to include one or more nucleic acids provided herein can be prokaryotic cells or eukaryotic cells. Examples of prokaryotic cells that can include one or more nucleic acids provided herein (e.g., a nucleic acid encoding an alpha chain and/or beta chain of a TCR provided herein, or a nucleic acid vector or viral vector provided herein) include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells.

Examples of eukaryotic cells that can include one or more nucleic acids provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein) include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, Chinese hamster ovary (CHO) cells, W138 BHK cells, COS-7 cells, and MDCK cells can be designed to include a nucleic acid provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein).

In some cases, a eukaryotic cell that includes a nucleic acid provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein) is an immune cell such as a T cell. Such cells can include an exogenous TCR, expressed from a nucleic acid provided herein, on their surface that has reactivity against the mutated form of ATM without having reactivity to the non-mutated form of ATM and/or an exogenous TCR, expressed from a nucleic acid provided herein, on their surface that has reactivity against the mutated form of ARID1A without having reactivity to the non-mutated form of ARID1A. A T cell can be any type of T cell and can be of any developmental stage, including CD4⁺ and/or CD8⁺ T cells. For example, the T cells can be helper T cells, e.g., Th1 and Th2 cells, CD8⁺ T cells (e.g., cytotoxic T cells), tumor infiltrating cells (TILs), memory T cells, naive T cells, cytotoxic T lymphocytes (CTLs), regulatory T cells, activated T cells, memory T cells, or natural killer cells. The T cell can be a cultured T cell, e.g., a primary T cell or a T cell from a cultured T cell line, e.g., Jurkat or Sup-T1. In some cases, the T cell can be isolated from a mammal such as a human. Mammalian T cells can be obtained, for example, from blood, bone marrow, lymph node, the thymus, or other tissues or fluids. T cells also can be enriched or purified.

In some cases, the T cell can be isolated from the mammal to be treated, i.e., the cells are autologous to the mammal (e.g., human), and modified to include a nucleic acid provided herein (e.g., a nucleic acid encoding an alpha chain and/or beta chain of a TCR provided herein, or a nucleic acid vector or viral vector provided herein) and comprise an exogenous TCR on the surface of the cell that is expressed from the nucleic acid. For example, for autologous cells, the immune cells can be obtained from the mammal's blood, cord blood, or bone marrow. In some cases, the T cells are heterologous to the mammal, i.e., the cells are not from the mammal to be treated.

In some cases, an immune cell such as a T cell (e.g., a human T cell) that includes a nucleic acid provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein) and comprises an exogenous TCR expressed from the nucleic acid, can lack expression of an endogenous alpha chain of a TCR and/or lack expression of an endogenous beta chain of a TCR. Any appropriate method can be used to generate T cells that lack expression of one or both chains of an endogenous TCR. For example, gene editing techniques such as those that involve using Clustered Regularly Interspaced Short Palindromic Repeats (CRISPR) technology or Transcription Activator-Like Effector Nuclease (TALEN) technology can be used to interfere with the expression of one or both chains of an endogenous TCR.

In some cases, an immune cell such as a natural killer (NK) cell that includes a nucleic acid provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein) and comprises an exogenous TCR expressed from the nucleic acid, can be engineered to express one or more of the CD3 chains of the TCR complex (e.g., the CD3ε, CD3γ, CD3 ζ, and optionally CD3δ). In such cases, an exogenous TCR can be expressed on the surface of the immune cell (e.g., a NK cell) in combination with the exogenously provided one or more CD3 chains of a CD3 complex.

In some cases, an immune cell such as a T cell (e.g., a human T cell) that includes a nucleic acid provided herein (e.g., a nucleic acid encoding an alpha chain and/or beta chain of a TCR having affinity for an ATM peptide and/or a nucleic acid encoding an alpha chain and/or beta chain of a TCR having affinity for an ARID1A peptide, or a nucleic acid vector or viral vector provided herein) and comprises an exogenous TCR expressed from the nucleic acid, expresses an endogenous TCR. In such cases, a portion of the TCRs present on the surface of such T cells can be endogenous TCRs, a portion of the TCRs present on the surface of such T cells can be exogenously provided TCRs (e.g., TCRs containing a combination of alpha and beta chain polypeptides described herein), and a portion of the TCRs present on the surface of such T cells can have one endogenous provided TCR chain and one exogenously provided TCR chain.

In some cases, the constant regions of the alpha and beta chains encoded by a nucleic acid provided herein can be engineered to include sequences that encode one or more cysteine residues to increase the pairing of the alpha and beta chains with each other when expressed within a cell. Examples of such cysteine residues include, without limitation, those described elsewhere (see, e.g., Kuball et al., Blood, 109:2331-2338 (2007)).

Any appropriate method can be used to introduce one or more nucleic acids provided herein (e.g., a vector containing a nucleic acid encoding the alpha chain and/or beta chain polypeptides of a TCR provided herein) into a cell (e.g., a host cell or an isolated cell). For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a cell (e.g., a host cell or an isolated cell) (see, e.g., Green and Sambrook, Molecular Cloning: A Laboratory Manual, 4th edition, Cold Spring Harbor Laboratory, NY (2012); Davis et al., Basic Methods in Molecular Biology (1986); and Neumann et al., EMBO J., 1:841 (1982)).

The immune cells (e.g., T cells) can be activated and expanded, before or after introduction of the nucleic acid. T-cell activation reagents are commercially available, including CTS™ Dynabeads™ CD3/28, MACS GMP ExpAct Treg beads (Miltenyi Biotec), MACS GMP TransAct CD3/28 beads (Miltenyi Biotec), and Expamer technology (Juno Therapeutics). See, for example, Wang and Riviere, Molecular Therapy Oncolytics, 3:16015 (2016). T cells can be expanded, for example, using a GE WAVE bioreactor system that includes a Cellbag Bioreactor and a rocking base, and which allows the cells to expand to more than 10⁷ cells/mL. In some cases, T cells can be expanded using Gas Permeable Rapid Expansion (G-Rex®) (Wilson Wolf Manufacturing), which includes a cell culture flask with a gas-permeable membrane at the base that allows cells to grow to a high density. T cells also can be expanded using a CliniMACS Prodigy system (Miltenyi Biotec), which uses a magnetic cell separation system, and a cell cultivation device. See, for example, Wang and Riviere, Molecular Therapy Oncolytics, 3:16015 (2016).

In some cases, the immune cells (e.g., T cells) that include, for example, a vector containing a nucleic acid encoding the alpha chain and/or beta chain polypeptides of a TCR provided herein, can be cryopreserved for use at a later time. For example, the cells can be cryopreserved with dimethyl sulfoxide (DMSO) and human serum albumin (HSA). In some cases, the cells can be cryopreserved with a balanced crystalloid solution (e.g., Plasma-Lyte 148), DMSO, and HSA.

In some cases, a nucleic acid provided herein, a vector provided herein, or an immune cell provided herein can be formulated as a pharmaceutical composition for administration to a mammal (e.g., a human) to treat a disorder, disease, or condition. For example, immune cells that comprise an exogenous TCR provided herein having affinity for an ATM peptide and/or an exogenous TCR provided herein having affinity for an ARID1A peptide can be formulated and used for cell therapy, e.g., adoptive cell therapy. In some cases, a nucleic acid, a vector, or an immune cell provided herein can be formulated for administration to a mammal (e.g., a human to treat cancer). In some cases, a pharmaceutical composition provided herein can include a pharmaceutically acceptable carrier such as a buffer, a salt, a surfactant, a sugar, a tonicity modifier, or combinations thereof. See, for example, Gervasi, et al., Eur. J. Pharmaceutics and Biopharmaceutics, Volume 131, pages 8-24 (2018). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include immune cells comprising an exogenous TCR provided herein having affinity for an ATM peptide (or nucleic acid or vector encoding an alpha and/or beta chain of a TCR) or an exogenous TCR provided herein having affinity for an ARID1A peptide (or nucleic acid or vector encoding an alpha and/or beta chain of a TCR) can be formulated to include a buffer (e.g., an acetate, citrate, histidine, succinate, phosphate, hydroxymethylaminomethane (Tris), or Plasma-lyte buffer).

In some cases, when a pharmaceutical composition is formulated to include immune cells, the formulation contains a sufficient number of cells to deliver about 1×10⁵ to about 1×10¹⁰ cells/kg body weight to the mammal. For example, the formulation contains a sufficient number of cells to deliver 1×10⁵ to about 1×10⁹ cells/kg body weight, 1×10⁵ to about 1×10⁸ cells/kg body weight, 2×10⁵ to about 2×10⁶ cells/kg body weight, or 1×10⁶ to about 1×10⁷ cells/kg body weight to the mammal.

In some cases, when a pharmaceutical composition is formulated to include one or more nucleic acids (e.g., vectors such as viral vectors) encoding an alpha and/or beta chain of a TCR provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid (e.g., a vector such as a viral vector) encoding an alpha and/or beta chain of a TCR per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid (e.g., a vector such as a viral vector) encoding a an alpha and/or beta chain of a TCR.

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more nucleic acids provided herein) can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing immune cells that include an exogenous TCR provided herein having affinity for an ATM peptide (or nucleic acid or vector encoding an alpha and/or beta chain of such a TCR), an exogenous TCR provided herein having affinity for an ARID1A peptide (or nucleic acid or vector encoding an alpha and/or beta chain of such a TCR), or both a TCR having affinity for a mutant ATM peptide and a TCR having affinity for an ARID1A peptide (or nucleic acid or vector encoding an alpha and/or beta chain of both of such TCRs) to a mammal (e.g., a human). For example, a composition (e.g., a pharmaceutical composition provided herein) containing immune cells that include an exogenous TCR having affinity for an ATM peptide, an exogenous TCR having affinity for an ARID1A peptide, or both a TCR having affinity for a mutant ATM peptide and a TCR having affinity for an ARID1A peptide provided herein (or nucleic acid or vector encoding an alpha and/or beta chain of one or both of such TCRs) can be administered to a mammal (e.g., a human) in need thereof to treat a disorder, disease, or condition. Any appropriate method can be used to administer a composition (e.g., a pharmaceutical composition provided herein) to a mammal (e.g., a human). For example, a composition (e.g., a pharmaceutical composition provided herein) containing immune cells that include an exogenous TCR provided herein having affinity for an ATM peptide and/or an exogenous TCR provided herein having affinity for an ARID1A peptide (or nucleic acid or vector encoding an alpha and/or beta chain of one or both of such TCRs) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) containing immune cells that include an exogenous TCR provided herein (or nucleic acid or vector encoding an alpha and/or beta chain of such a TCR) can be adjusted for the mammal being treated.

Effective doses of a composition containing immune cells that include an exogenous TCR provided herein having affinity for an ATM peptide, and/or an exogenous TCR provided herein having affinity for an ARID1A peptide (or nucleic acid or vector encoding an alpha and/or beta chain of one or both of such TCRs), or both a TCR having affinity for a mutant ATM peptide and a TCR having affinity for an ARID1A peptide can vary depending on the severity of the disorder, the route of administration, the age and general health condition of the subject, excipient usage, the possibility of co-usage with other therapeutic treatments such as use of other agents, and the judgment of the treating physician.

In some cases, an effective amount of a composition containing immune cells comprising an exogenous TCR provided herein having affinity for an ATM peptide and/or an exogenous TCR provided herein having affinity for an ARID1A peptide (or nucleic acid or vector encoding an alpha and/or beta chain of one or both of such TCRs) can be an amount that reduces the activity of the target antigen within a mammal without producing significant toxicity to the mammal. For example, an effective amount of immune cells that comprise an exogenous TCR provided herein can be from about 1×10⁵ to about 1×10¹⁰ cells/kg body weight. For example, the cells can be administered to the mammal at a range of 1×10⁵ to about 1×10⁹ cells/kg body weight, 1×10⁵ to about 1×10⁸ cells/kg body weight, 2×10⁵ to about 2×10⁶ cells/kg body weight, 1×10⁶ to about 1×10⁷ cells/kg body weight.

The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of immune cells that include an exogenous TCR provided herein having affinity for an ATM peptide and/or an exogenous TCR provided herein having affinity for an ARID1A peptide (or nucleic acid or vector an alpha and/or beta chain of one or both of such TCRs) can be any amount that maintains a fairly steady state of reduced target antigen activity within a mammal without producing significant toxicity to the mammal. For example, the frequency of administration of immune cells provided herein can be from about twice daily to about once a week. In some cases, the frequency of administration of immune cells provided herein can be daily. The frequency of administration of immune cells provided herein can remain constant or can be variable during the duration of treatment. A course of treatment with a composition containing immune cells provided herein can include rest periods. For example, a composition containing immune cells provided herein can be administered daily over a two-week period followed by a one-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing immune cells that include an exogenous TCR provided herein having affinity for an ATM peptide, an exogenous TCR provided herein having affinity for an ARID1A peptide, or both a TCR having affinity for a mutant ATM peptide and a TCR having affinity for an ARID1A peptide (or nucleic acid or vector encoding the alpha and/or beta chains of one or both of such TCRs) can be any appropriate duration that reduces the activity of the target antigen within a mammal without producing significant toxicity to the mammal. In some cases, the effective duration can vary from several weeks to several years (e.g., 5, 10, 15, or more years). Multiple factors can influence the actual effective duration used for a particular treatment. For example, an effective duration can vary with the frequency of administration, effective amount, use of multiple treatment agents, route of administration, and severity of the condition being treated.

In some cases, a composition containing immune cells that include an exogenous TCR provided herein having affinity for an ATM peptide, an exogenous TCR provided herein having affinity for an ARID1A peptide, or both a TCR having affinity for a mutant ATM peptide and a TCR having affinity for an ARID1A peptide (or nucleic acid or vector encoding the alpha and/or beta chains of one or both of such TCRs) can be administered as part of a combination treatment, such as simultaneously with or sequentially with, in any order, another therapeutic agent, such as a cytokine (e.g., IL-2), antibody (e.g., tocilizumab, which blocks IL-6 activity), an immune checkpoint inhibitor (e.g., one or more of an anti-PD-1 antibody, an anti-PDL-1 antibody, or an anti-CTLA-4 antibody), or a chemotherapeutic agent.

The invention will be further described in the following examples, which do not limit the scope of the invention described in the claims.

### EXAMPLES

To confirm the specificity and activity of nucleic acid encoding TCRs against ATM and ARID1A, each of the codon optimized nucleic acids were introduced into donor T cells. The chimeric TCR-transduced T cells (TRBV9*01+TRAV14/DV4 chimeric TCR) were co-cultured with dendritic cells expressing wildtype ATM or mutated forms of ATM (G2695V, G2695A, G2695C, or G2695S). The chimeric TCR-transduced T cells (TRBV25-1+TRAV20 chimeric TCR) were co-cultured with dendritic cells expressing wildtype ARID1A or ARID1A H1960fs. After overnight incubation, the supernatant was assessed for production of interferon-gamma using an ELISA. It was found that the introduced nucleic acids encoding TCRs conferred mutation reactivity without reactivity against the wild-type form of ATM and ARID1A. See FIGS. 9A and 9B, respectively.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated human immune cell comprising an exogenous TCR having affinity for a human AT-rich interactive domain-containing protein 1A (ARID1A) peptide comprising a frameshift at the position corresponding to 1960 in the human ARID1A protein in the context of HLA A*02:01, wherein said position corresponding to 1960 of said peptide is a proline instead of a histidine, said exogenous TCR comprising an alpha chain and a beta chain,
said alpha chain comprising the amino acid sequences set forth in SEQ ID NO: 32, SEQ ID NO:33, and SEQ ID NO:34; and
said beta chain comprising the amino acid sequences set forth in SEQ ID NO: 23, SEQ ID NO:24, and SEQ ID NO:25.

2. The immune cell claim 1, wherein said alpha chain comprises an amino sequence having at least 80 percent identity to the amino acid sequence set forth in SEQ ID NO:40 or SEQ ID NO:44.

3. The immune cell of claim 1, wherein said beta chain comprises an amino acid sequence having at least 80 percent identity to the amino acid sequence set forth in SEQ ID NO:31 or SEQ ID NO:42.

4. The immune cell of claim 1, wherein said immune cell is a T cell.

5. The immune cell of claim 1, wherein expression of the endogenous TCR alpha and beta chain coding sequences are downregulated in said T cell.

6. A nucleic acid molecule comprising (a) a nucleic acid sequence encoding an alpha chain of a TCR having affinity for a human ARID1A peptide comprising a frameshift at the position corresponding to 1960 in the human ARID1A protein in the context of HLA A*02:01, and (b) a nucleic acid sequence encoding a beta chain of said TCR, wherein said position corresponding to 1960 of said peptide is a proline instead of a histidine,
said alpha chain comprising the amino acid sequences set forth in SEQ ID NO: 32, SEQ ID NO:33, and SEQ ID NO:34; and
said beta chain comprising the amino acid sequences set forth in SEQ ID NO: 23, SEQ ID NO:24, and SEQ ID NO:25.

7. The nucleic acid of claim 6, wherein said nucleic acid is a vector.

8. The nucleic acid of claim 7, wherein said vector is a viral vector.

9. A method for treating a mammal having cancer, said method comprising administering, to said mammal, (a) a population of cells or (b) nucleic acid, wherein said population of cells comprises at least one immune cell comprising said nucleic acid, wherein said immune cell comprises an exogenous TCR having affinity for a human ARID1A peptide comprising a frameshift at the position corresponding to 1960 in the human ARID1A protein in the context of HLA A*02:01, wherein said position corresponding to 1960 of said peptide is a proline instead of a histidine, and wherein said nucleic acid comprises a nucleic acid sequence encoding an alpha chain of said TCR and a nucleic acid sequence encoding a beta chain of said TCR,
said alpha chain comprising the amino acid sequences set forth in SEQ ID NO: 32, SEQ ID NO:33, and SEQ ID NO:34; and
said beta chain comprising the amino acid sequences set forth in SEQ ID NO: 23, SEQ ID NO:24, and SEQ ID NO:25.

10. The method of claim 9, wherein said mammal is a human.

11. The method of claim 10, wherein said method comprises administering said nucleic acid to said mammal.

12. The method of claim 11, wherein said nucleic acid is a vector.

13. The method of claim 12, wherein said vector is a viral vector.

14. The method of claim 9, wherein said method comprises administering said population of cells to said mammal.

15. The method of claim 10, wherein said method comprises administering said population of cells to said human.

16. The method of claim 15, wherein said cells are autologous to said human.
